(19) United States
(12) Patent Application Publication
(10) Pub. No.: US 2023/0149073 A1
(43) Pub. Date: May 18, 2023

(54) METHOD AND DEVICE FOR PROBE NAVIGATION OF AN ABLATION SYSTEM

(71) Applicant: AngioDynamics, Inc., Latham, NY (US)

(72) Inventors: Hayley FORREST, Natick, MA (US); Rachel BEAN, Seekonk, MA (US)

(73) Assignee: AngioDynamics, Inc., Latham, NY (US)

(21) Appl. No.: 18/055,004

(22) Filed: Nov. 14, 2022

Related U.S. Application Data
(60) Provisional application No. 63/279,432, filed on Nov. 15, 2021.

(51) Int. Cl. A61B 18/14; A61B 6/00; A61B 34/10; A61B 8/00

(52) U.S. Cl. CPC A61B 18/1492; A61B 6/5211; A61B 8/462; A61B 34/10; A61B 2034/107

(57) ABSTRACT

A system for preparing an ablation procedure is disclosed. The system comprises a generator for generating electrical pulses, a plurality of probes for delivering the electrical pulses to a target tissue, one or more sensors for indicating a position of the probes, a display device, and a processor. The processor is configured to receive an image of the target tissue, obtain a planned probe arrangement for the plurality of probes, identify a projected position for an origin probe based on the image and the planned probe arrangement, and determine a real-time position of the origin probe based on signals from the sensors. The processor is further configured to display the projected position and the real-time position for the origin probe superimposed over the image on a display device, and determine a placed position of the origin probe based on the real-time position.

### CROSS-REFERENCE TO RELATED APPLICATIONS

This application claims the benefit of priority to U.S. Provisional Application No. 63/279,432 entitled “Method and Device for Probe Navigation of an Ablation System,” filed Nov. 15, 2021, which is incorporated herein by reference in its entirety.

### TECHNICAL FIELD

The present disclosure relates generally to methods, systems, and apparatuses related to performing an irreversible electroporation (IRE) procedure on a patient. More particularly, the present disclosure relates to methods and systems for planning an IRE procedure and preparing for an IRE procedure. The disclosed techniques may be applied to, for example, ablation of tumors within a tissue of a human patient.

### BACKGROUND

Irreversible electroporation (IRE) is a non-thermal, minimally invasive surgical technique to ablate undesirable tissue, e.g., tumor tissue. The technique is easy to apply, can be monitored and controlled, is not affected by local blood flow, and does not require the use of adjuvant drugs. The minimally invasive procedure involves placing needle-like electrodes into or around a target tissue to deliver a series of short and intense electric pulses that induce structural changes in the cell membranes. The electric pulses subject cells in the target tissue to an electric field, which alters their native transmembrane potential and causes formation of nanoscale defects that facilitate macromolecule transport and disruption of the membrane’s ability to maintain cellular environment homeostasis. When the strength of the pulsing protocol is sufficient, the cell cannot recover from these defects and dies. IRE is particularly useful in scenarios requiring precision and conservation of the extracellular matrix, blood flow, nerves, and other tissue.

However, conventional systems for planning and performing IRE procedures have several shortcomings that prevent an operator from completing the procedure in an efficient and effective manner. One major difficulty is that planning the placement of probes is a complex and time-consuming process. Electrodes must be placed in a manner that results in a lethal dose of ablation to the entire target tissue while sparing surrounding healthy tissue from serious damage. Because the probe placement depends on the location, shape, and size of the target tissue, each case must be individually planned and may require a great deal of trial and error. Furthermore, delicate structures present adjacent the target tissue may further complicate planning.

Another difficulty for surgeons is placement of the electrodes into the tissue in compliance with the ablation plan to target the desired tissue. Placement of electrodes in an accurate manner is extremely time consuming and requires a great amount of expertise. In many cases, electrode placement can take several hours to complete adequately. The distance between electrodes, the angle of insertion, the depth, and the presence of interfering structures in the patient anatomy can all impact the flow of electricity. Thus, improper placement may result in failure to properly ablate the target tissue and/or collateral damage to the surrounding healthy tissue. Further, in conventional systems, surgeons must measure the distances between electrodes after placement and manually input this information to estimate the electric field that may be produced between the electrodes, which is also a time-consuming and error-prone process.

As such, it would be advantageous to have a tool for facilitating and/or partially automating planning of IRE procedures in a manner that enables context-aware treatment with greater overall efficacy and reduced planning time. Additionally, it would be advantageous to have a tool for facilitating guided placement of IRE electrodes with greater accuracy and reduced preparation time.

### SUMMARY

A system for preparing an ablation procedure is provided. The system comprises: a generator configured to generate at least one electrical pulse for delivery to a target tissue; a plurality of probes operably connected to the generator, the plurality of probes configured to deliver the at least one electrical pulse to the target tissue; one or more sensors configured to indicate a position of each probe of the plurality of probes; a display device; a processor operably connected to the generator and the display; and a non-transitory, computer-readable medium storing instructions that, when executed, cause the processor to: receive an image of the target tissue, obtain an ablation plan relating to the target tissue, the ablation plan comprising a planned probe arrangement including a planned position for each of the plurality of probes, identify, based on the image and the planned probe arrangement, a projected position for an origin probe of the plurality of probes, determine, based on one or more signals from the one or more sensors, a real-time position of the origin probe, display, on the display device, the projected position and the real-time position for the origin probe superimposed over the image, and determine a placed position of the origin probe based on the real-time position.

According to some embodiments, the processor is operably connected to an imaging device, and the instructions to receive an image of the target tissue comprise instructions that, when executed, cause the processor to receive the image of the target tissue from the imaging device.

According to some embodiments, the instructions, when executed, further cause the processor to: compare the placed position of the origin probe to the projected position of the origin probe, and modify the ablation plan based on the comparison. According to additional embodiments, the instructions to modify the ablation plan comprise instructions that, when executed, cause the processor to update the planned position for one or more additional probes of the plurality of probes based on the comparison. According to additional embodiments, the instructions to modify the ablation plan comprise instructions that, when executed, cause the processor to update one or more treatment parameters of the ablation plan based on the comparison.

According to some embodiments, the system further comprises one or more input devices, and the instructions to obtain an ablation plan comprise instructions that, when executed, cause the processor to: identify, based on the image, a proposed target zone comprising at least a portion of the target tissue, display, on the display device, the proposed target zone superimposed over the anatomical image, receive, by the one or more input devices, target feedback related to the proposed target zone, modify the proposed target zone based on the target feedback to define a selected target zone, and generate the ablation plan, wherein the ablation plan comprises the selected target zone. According to additional embodiments, the instructions, when executed, further cause the processor to: measure one or more distances in the selected target zone based on the image, and define a location of the selected target zone based on the image, wherein the ablation plan is based on the one or more distances and the location of the selected target zone. According to additional embodiments, the instructions to generate the ablation plan comprise instructions that, when executed, further cause the processor to: identify, based on the selected target zone, a proposed probe arrangement comprising a proposed position for each of the plurality of probes, display, on the display device, the proposed probe arrangement superimposed over the image, receive, by the one or more input devices, probe feedback related to the proposed probe arrangement, modify the proposed probe arrangement based on the probe feedback to define a selected probe arrangement, and update the ablation plan based on the selected probe arrangement, wherein the planned probe arrangement of the ablation plan comprises the selected probe arrangement. According to further embodiments, the probe feedback comprises one or more of: an indication of one or more of the plurality of probes to be removed from the proposed probe arrangement; an indication to add one or more additional probes to the plurality of probes in the proposed probe arrangement; an indication to adjust the proposed position of one or more of the plurality of probes; and an indication of approval of the proposed probe arrangement. According to further embodiments, the ablation plan further comprises a planned series of pulses to be emitted between the plurality of probes, and the instructions to update the ablation plan based on the selected probe arrangement comprise instructions that, when executed, cause the processor to update the planned series of pulses based on the selected probe arrangement.

An ablation system is also provided. The ablation system comprises a non-transitory, computer-readable medium storing instructions that, when executed by a processor, cause the processor to: receive an image of a target tissue; obtain a planned probe arrangement including a planned position for each of a plurality of probes; identify, based on the image and the planned probe arrangement, a projected position for a first probe of the plurality of probes; determine, based on one or more signals from one or more sensors, a real-time position of the first probe; display, via a display device, the projected position and the real-time position for the first probe superimposed over the image; and determine a placed position of the first probe based on the real-time position.

According to some embodiments, the instructions, when executed, further cause the processor to: compare the placed position of the first probe to the projected position of the first probe, and modify a treatment plan based on the comparison. According to additional embodiments, the instructions to modify the treatment plan comprise instructions that, when executed, cause the processor to update the planned position for one or more additional probes of the plurality of probes based on the comparison. According to additional embodiments, the instructions to modify the treatment plan comprise instructions that, when executed, cause the processor to update one or more treatment parameters of the treatment plan based on the comparison.

A system for planning an ablation procedure is also provided. The system comprises: a display device; a processor operably connected to the display device and the one or more input devices; and a non-transitory, computer-readable medium storing instructions that, when executed, cause the processor to: receive an image of a patient comprising a target tissue, receive, by one or more input devices, target feedback related to the identification of a target zone, identify a planned position for a first probe of a plurality of probes relative to the target zone, determine, based on one or more signals from a sensor, a real-time position of the first probe, display, on the display device, the planned position and the real-time position for the first probe superimposed over the image of the target tissue and the target zone, and determine a placed position of the first probe based on the real-time position.

According to some embodiments, the system further comprises: a generator configured to generate at least one electrical pulse for delivery to a target tissue; and the plurality of probes operably connected to the generator, wherein the plurality of probes are configured to deliver the at least one electrical pulse to the target tissue to irreversibly electroporate substantially all of the target tissue in the target zone.

According to some embodiments, the sensor comprises an electromagnetic sensor. According to additional embodiments, the system further comprises the electromagnetic sensor and an electromagnetic generator operably connected to the processor. According to further embodiments, the electromagnetic generator is arranged and configured to generate an electromagnetic field in a region including the target tissue. According to still further embodiments, the instructions to determine a real-time position of the first probe comprise instructions that, when executed, cause the processor to: receive the one or more signals from the electromagnetic sensor, wherein the one or more signals are indicative of a characteristic of the electromagnetic field sensed by the sensor, and determine a location of the electromagnetic sensor based on the one or more signals, wherein the location of the electromagnetic sensor is indicative of the real-time position of the first probe.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. Various aspects of at least one example are discussed below with reference to the accompanying drawings, which are not intended to be drawn to scale. In the drawings:

FIG. 1 depicts a block diagram of an exemplary user interface system in accordance with an embodiment.

FIG. 2 depicts a block diagram of an illustrative system for planning an IRE procedure in accordance with an embodiment.

FIG. 3 depicts a flow diagram of an illustrative computer-implemented method for planning an IRE procedure in accordance with an embodiment.

FIG. 4 depicts an anatomical image displayed on a user interface in accordance with an embodiment.

FIG. 5 depicts an exemplary user interface stage for displaying a predicted target zone in accordance with an embodiment.

FIG. 6 depicts an exemplary user interface stage for obtaining target feedback related to a predicted target zone in accordance with an embodiment.

FIG. 7 depicts an exemplary user interface stage comprising a selected target zone in accordance with an embodiment.

FIG. 8 depicts a flow diagram of an illustrative computer-implemented method for generating an ablation plan comprising a probe arrangement in accordance with an embodiment.

FIG. 9 depicts an exemplary user interface stage for planning a probe arrangement in accordance with an embodiment.

FIG. 10 depicts an exemplary user interface stage comprising an electric field strength map in accordance with an embodiment.

FIG. 11 depicts a block diagram of an illustrative system for placing electrodes for an IRE procedure in accordance with an embodiment.

FIG. 12 depicts a flow diagram of an illustrative computer-implemented method for preparing an IRE procedure in accordance with an embodiment.

FIG. 13 depicts an exemplary user interface stage for displaying a placed position of an origin probe in accordance with an embodiment.

FIG. 14 depicts an exemplary user interface stage for guiding placement of a secondary probe in accordance with an embodiment.

FIG. 15 depicts an exemplary user interface stage for reviewing placed probe measurements in accordance with an embodiment.

FIG. 16 illustrates a block diagram of an exemplary data processing system in which embodiments are implemented.

### DETAILED DESCRIPTION

This disclosure is not limited to the particular systems, devices and methods described, as these may vary. For example, while the present disclosure is described relative to IRE, it is to be understood that the present disclosure is equally applicable to other non-thermal ablation techniques, such as, reversible electroporation, electro-chemotherapy (ECT), high frequency irreversible electroporation (HFIRE), pulsed field ablation (PFA), pulsed electric fields (PEF), and the like. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

All percentages, parts and ratios of a composition are based upon the total weight of the composition and all measurements made are at about 25° C., unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by ⅒ of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “patient” and “subject” are interchangeable and refer to any living organism which contains neural tissue. As such, the terms “patient” and “subject” may include, but are not limited to, any non-human mammal, primate or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child or infant.

The term “tissue” refers to any aggregation of similarly specialized cells which are united in the performance of a particular function.

The term “disorder” is used in this disclosure to mean, and is used interchangeably with, the terms “disease,” “condition,” or “illness,” unless otherwise indicated.

The term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

In the present disclosure various systems, components, and methods related to an irreversible electroporation (IRE) system and a user interface provided by the IRE system. Each of the systems, components, and methods disclosed herein provides one or more advantages over conventional systems, components, and methods. As discussed herein, it would be advantageous to have a system for identifying a target treatment zone and proposing a probe arrangement for treating the target treatment zone. Furthermore, it would be advantageous to have a system for guiding placement of one or more probes with respect to a target treatment zone according to an ablation plan.

### IRE Interface System

The IRE systems utilized by the systems herein are first described in greater detail. Such IRE systems may utilize user interfaces that dynamically display information provided by an operator and/or the IRE system during setup, planning, and implementation stages of an IRE procedure. As a result, an operator can plan and implement more effective IRE procedures and monitor various IRE pulse parameters in real-time, thereby providing greater control and customization of the IRE treatment settings. In various embodiments, the user interfaces may dynamically display a pulse parameters table for textual display of numerical values indicating a voltage, a pulse length, a number of pulses, and/or a distance for an active probe pair of a selected probe array. The pulse parameter table may further indicate a polarity of the active probe pair. The user interfaces may further provide a probe placement grid for graphical display of the user selected probe array in relation to a lesion zone and/or a target ablation zone. Each probe of the selected probe array may be represented in the probe placement grid as a probe grid icon and/or a probe number. In some embodiments, an active probe pair of the selected probe array may be indicated by a dashed line connecting the probe grid icons of the active probe pair. In additional embodiments, an active probe pair of the selected probe array may be indicated by a shape that links or surrounds the active probe pair. In further embodiments, the active probe pair may be displayed in a first color while the remainder of the probes in the selected probe array may be displaying in a second color. In some embodiments, the polarity of the active probe pair may be indicated by an arrow of the dashed line pointing to a negative probe of the active probe pair to depict the directional flow of current. In some embodiments, the distance between the active probe pair may be indicated by a distance value positioned over the dashed line and/or by a spacing between the probe grid icons of the active probe pair. In some embodiments, modification of values displayed in the pulse parameters table may be dynamically reflected in the graphical depiction of the selected probe array in the probe placement grid. In additional embodiments, as opposed to one or more probe pairs, the user interface may depict a single insertion device (SID) including two electrodes spaced apart on a single probe.

As described, the user interface may dynamically display a pulse generation table for textual display of numerical values indicating a voltage, a pulse duration, and a number of pulses for pulse delivery by an active probe pair of a user selected probe array. The user interface may include a probe pair status grid for graphical display of the user selected probe array in relation to a lesion zone and a target ablation zone. In some embodiments, an electrical results chart may be provided by the user interface for graphical display of the pulse delivery by the active probe pair based on the numerical values for the voltage, the pulse duration, and the number of pulses for the active probe pair specified within the pulse generation table. In some embodiments, the pulse generation table may be updated during and after implementation of the pulse delivery to numerically indicate data graphically depicted in the electrical results chart.

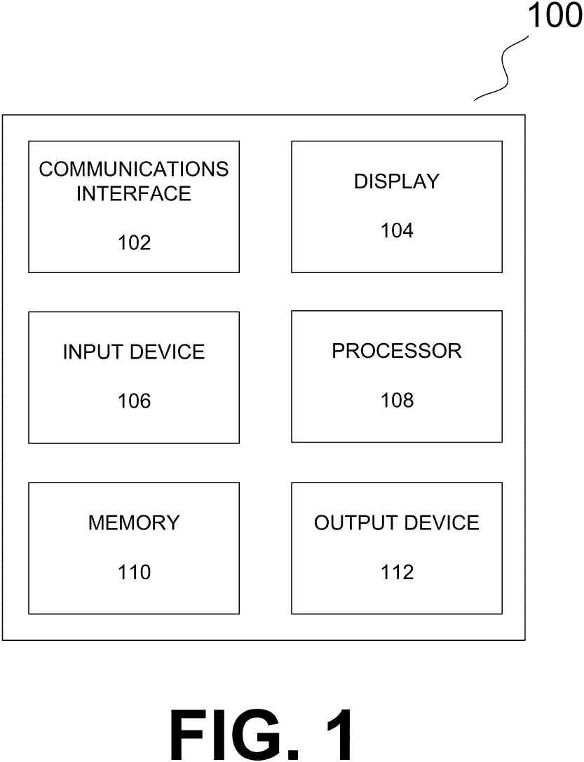

Referring now to FIG. 1, a block diagram of an exemplary user interface system is depicted in accordance with an embodiment. The user interface system 100 may be coupled to and/or can be a component of a system that provides irreversible electroporation (IRE) and/or other electroporation-based therapies (EBTs), e.g., electrochemotherapy and/or electrogenetherapy. For example, the user interface system 100 may be coupled to an IRE system configured to generate and deliver electric pulses through one or more probes to a patient for therapeutic purposes. In some embodiments, the IRE system is configured to perform an ablation procedure involving the delivery of a series of high voltage direct current electrical pulses between two or more electrodes (e.g., two, three, four, five, six, or more electrodes) placed within or around a targeted region for ablation (i.e., a target ablation zone). In some embodiments, a single electrode may be positioned on a single probe, and a user may place several probes in or near a treatment site, thereby forming one or more active probe pairs. In additional embodiments, two or more electrodes may be positioned on a single probe (e.g., a SID). Additional details and description of IRE systems are disclosed in U.S. Pat. Application No. 13/630,135, now issued as U.S. Pat. No. 9,078,665, and U.S. Pat. Application No. 15/565,625, each of which is incorporated herein by reference in its entirety.

The user interface system 100 may provide a graphical display (i.e., a user interface) to a surgeon or other user or operator of the IRE system. In some embodiments, the user interface may enable the user to specify one or more parameters for an IRE procedure, i.e., treatment parameters. In some embodiments, the user interface may enable the user to begin, pause, restart, or stop the IRE procedure. In some embodiments, the user interface may provide real-time information to the user for monitoring the IRE procedure. As shown in FIG. 1, the user interface system 100 may comprise a communications interface 102, a display 104, one or more input devices 106, a control unit comprising a processor 108 and a memory unit 110, and one or more output devices 112.

The communications interface 102 may provide communication links between the user interface system 100 and one or more remote devices (not depicted in FIG. 1). In some embodiments, the communications interface 102 further provides communications links between the user interface system 100 and an IRE system. Furthermore, any of the components of the user interface system may be situated remotely and the communications interface 102 may enable communication thereto. In some embodiments, the communications interface 102 may enable communication with one or more remote devices using, for example, Wi-Fi, a cellular communications standard, or Bluetooth. However, the communications interface 102 may utilize wired and/or wireless links according to any known wired or wireless communication standard or protocol as would be apparent to a person having an ordinary level of skill in the art.

The display 104 may be a visual display configured to render visual information, which may include any graphical or textual information as described herein. In some embodiments, the display 104 is a touchscreen or a touch-sensitive display. In some embodiments, the display 104 is wireless display remotely located from other components of the user interface system 100.

The one or more input devices 106 may comprise any number and type of input devices configured to receive and/or process input from a user. For example, the input devices 106 may include a mouse, a trackpad, a keyboard, a touchscreen, a microphone, a foot pedal, one or more knobs, one or more sliders, one or more switches, one or more user interaction components, and the like. However, any type of input device 106 as would be known to a person having an ordinary level of skill in the art may be utilized herein.

The one or more output devices 112 may comprise any number and type of output devices configured to generate and/or transmit outputs to an external device and/or a user. For example, the output devices 112 may include visible, audible, and/or tactile (e.g., vibrational) outputs. In some embodiments, the output devices 112 include one or more probes used for a specified IRE procedure.

The control unit may include a processor 108 for executing instructions stored in the memory unit 110. In some embodiments, the processor 108 is configured to control and direct operation of various components of the user interface system 100. For example, the processor 108 may control the operation of the communications interface 102, the display 104, the input devices 106, and the output devices 112. In some embodiments, the memory unit 110 is a non-transitory, computer-readable medium capable of being read by the processor 108 to execute the instructions thereon.

It should be understood that the communications interface 102, the display 104, the input devices 106, and the output devices 112 may be implemented in hardware, software, or any combination thereof. Furthermore, the user interface system 100 may include additional modules, components, or devices implemented in hardware, software, or any combination thereof and not shown in FIG. 1 to facilitate communication with remote devices, receiving input signals from a user or the IRE system, and/or presentation of visual or audible information to the user. For example, the user interface system 100 may further comprise additional controllers, interfaces, and/or electrical components to enable function of the user interface system 100, e.g., display controllers, input device interfaces, and/or output device interfaces.

As further described herein, the user interface system 100 may operate as part of an IRE system. For example, the user interface system 100 may provide a user interface that may be dynamically updated based on inputs provided by the user for setting parameters for an IRE procedure and/or based on information provided by the IRE system. In some embodiments, the user interface is also dynamically updated based on real-time delivery of pulses generated and applied in accordance with set treatment parameters.

In some embodiments, the user interface system 100 may be used for preparing and/or controlling an IRE procedure. For example, the user interface system may be used to establish parameters for carrying out, controlling, and/or monitoring the IRE procedure. The parameters (i.e., treatment parameters) may be pre-stored or specified by the user and stored (e.g., within the memory unit 110) thereafter. Accordingly, the IRE procedure may be carried out by the IRE system in accordance with the stored parameters. For example, electrical pulses may be generated and delivered to the patient through one or more probes based on the stored parameters. The user interface further enables the user to control (e.g., initiate) and monitor the pulse delivery. In some embodiments, the user may define and/or modify pulse parameters and other IRE treatment parameters using the user interface system 100. Furthermore, data regarding pulse delivery may be provided to the user interface system 100 during or after pulse delivery. Such data may be displayed or otherwise represented to the user as real-time results on the user interface.

In some embodiments, the user interface includes a plurality of operational modes provided based on direction from the processor 108. For example, based on the selected mode, the processor 108 and/or a graphics controller may retrieve a corresponding set of graphics from the memory unit 110 for presentation on the display 104. In some embodiments, any of the plurality of operational modes may be selected by the user through the touchscreen display 104 and/or the input devices 106.

It should be understood that additional information and/or data may be presented on the user interface from a variety of sources. In some embodiments, the communications interface 102 may receive data from one or more remote computing devices, databases (e.g., patient data databases), networks (e.g., a local hospital network), and/or cloud network or storage systems for display and/or use by the user interface. For example, remotely stored patient data (e.g., historical data or a data related to the current patient) may be retrieved and imported by the user interface system 100. Additional details and description of user interface systems and IRE systems are disclosed in U.S. Pat. Application No. 10/608,660, now issued as U.S. Pat. No. 7,435,236, U.S. Pat. Application No. 12/751,826, and U.S. Pat. Application No. 16/162,953, each of which is incorporated herein by reference in its entirety.

### Systems for Improved Planning of IRE Procedures

As discussed herein, IRE procedures provide electrical stimulation to a target tissue (e.g., a tumor) in order to induce permanent and lethal nanopores in the cell membrane, thereby disrupting cellular homeostasis and causing cell death. IRE is particularly useful in scenarios requiring precision and conservation of the extracellular matrix, blood flow, nerves, and other tissue. Accordingly, developing an ablation plan is a complex process with many factors that may be difficult and/or time-consuming to prepare without guidance. As such, it would be advantageous to have a tool for facilitating and/or partially automating planning of IRE procedures in a manner that enables context-aware treatment with greater overall efficacy and reduced planning time.

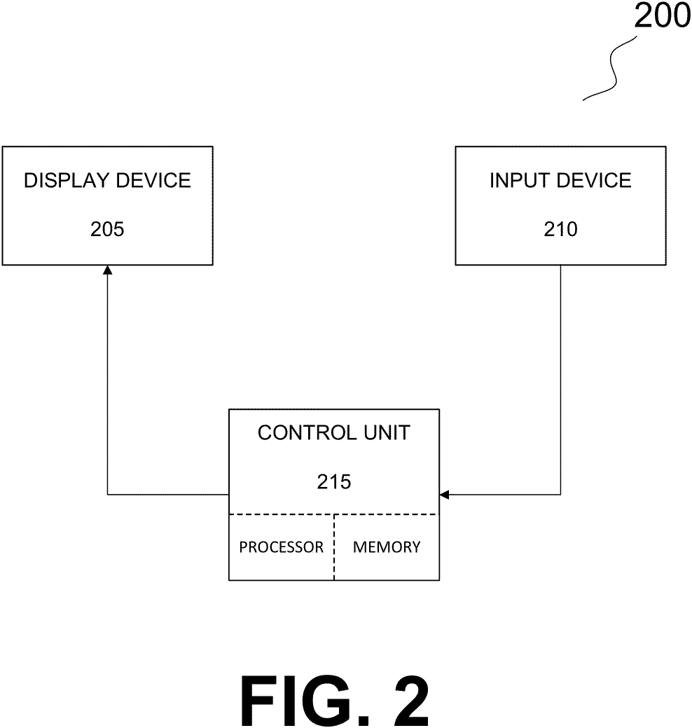

Turning now to FIG. 2, a block diagram of an illustrative system for planning an IRE procedure is depicted in accordance with an embodiment. The system 200 comprises a display device 205, at least one input device 210, and a control unit 215 comprising a processor and a memory. The display device 205 and the at least one input device 210 are each in electrical communication with the control unit 215.

The display device 205 may be a visual display configured to render visual information, which may include any graphical or textual information as described herein. For example, the display device 205 may present a user interface to a user that includes graphical or textual depictions of information related to an IRE procedure. As further described herein, the user interface may include several stages, i.e., different panels that are presented at different steps of planning. In some embodiments, the display device 205 is a touchscreen or a touch-sensitive display. In some embodiments, the display device 205 is a wireless display remotely located from other components of the system 200. The display device 205 may be a display, such as display 104 as described herein with respect to FIG. 1, and may comprise any of the features and/or functions as described with respect to the display 104.

The at least one input device 210 may comprise any number and type of input devices configured to receive and/or process input from a user. For example, the input device 210 may include a mouse, a trackpad, a keyboard, a touchscreen, a microphone, a foot pedal, one or more knobs, one or more sliders, one or more switches, one or more user interaction components, and the like. However, any type of input device 210 as would be known to a person having an ordinary level of skill in the art may be utilized herein. It should be understood the input device 210 may be an input device 106 as described herein with respect to FIG. 1 and may comprise any of the features and/or functions as described with respect to the input devices 106.

The control unit 215 may be in electrical communication with the display device 205 and the at least one input device 210 in order to enable a user to plan an IRE procedure. In some embodiments, the control unit 215 includes a processor and a memory such as a non-transitory, computer-readable medium storing instructions for presenting a user interface and preparing an ablation plan for an IRE procedure. It should be understood that the control unit 215 may comprise any number of components of the processor 108, the memory 110, and/or the user interface system 100 as described herein with respect to FIG. 1 (e.g., the communications interface 102 and/or the one or more output devices 112) and may comprise any of the features and/or functions as described with respect to the user interface system 100.

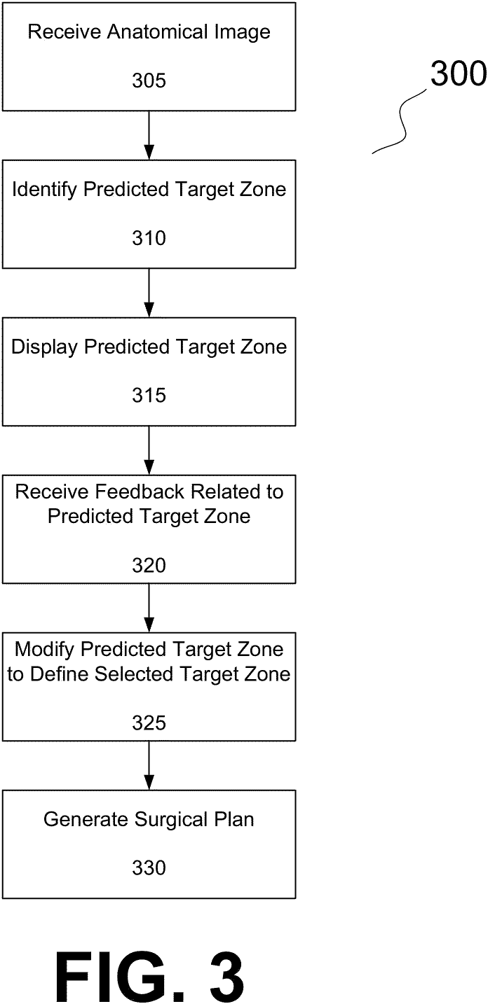

Turning now to FIG. 3, a flow diagram of an illustrative computer-implemented method for planning an IRE procedure by the system 200 is depicted in accordance with an embodiment. For example, the method 300 may be carried out by the processor of the control unit 215 upon execution of the instructions stored on the memory. The method 300 comprises receiving 305 an anatomical image of a patient, identifying 310 a predicted target zone for the IRE procedure, displaying 315 the predicted target zone on the display device 205, receiving 320 feedback related to the predicted target zone, modifying 325 the predicted target zone based on the feedback to define a selected target zone, and generating 330 an ablation plan based on the selected target zone.

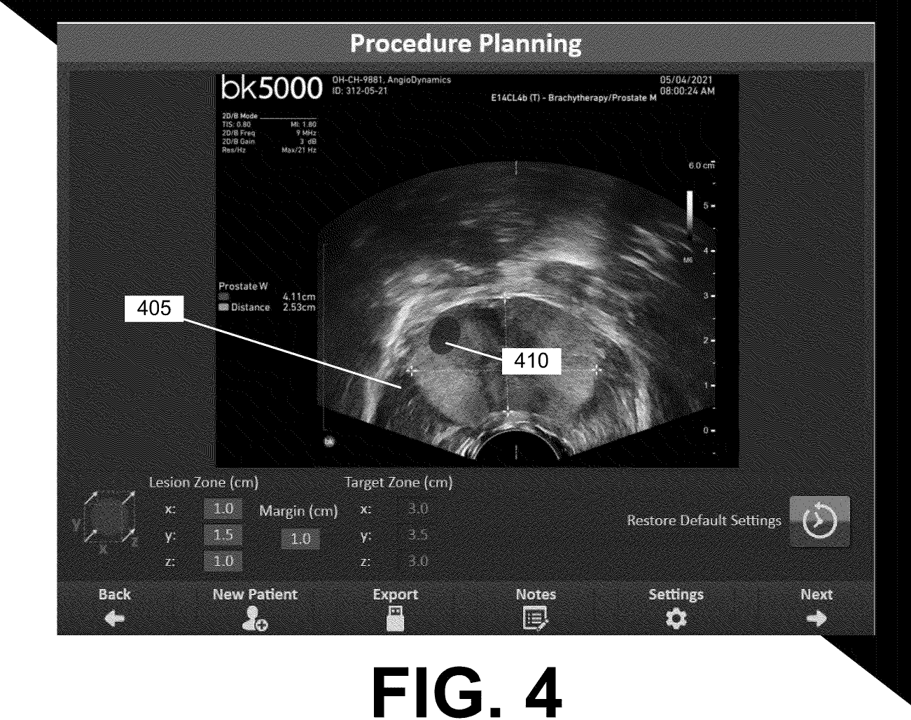

In some embodiments, the anatomical image is a medical image of the patient. The anatomical image may be captured by a variety of imaging modalities. For example, FIG. 4 depicts an anatomical image 405 displayed on a user interface in accordance with an embodiment. As shown, the anatomical image 405 may be an ultrasound image captured by an ultrasound imaging device. In another example, the anatomical image may be a computerized tomography (CT) image captured by a CT imaging device. In another example, the anatomical image may be a positron-emission tomography (PET) image captured by a PET imaging device. In another example, the anatomical image may be an x-ray image captured by an x-ray imaging device. However, it should be understood that any imaging modality appropriate for identifying a target tissue may be utilized herein as would be known to a person having an ordinary level of skill in the art.

In some embodiments, the anatomical image is received 305 from an imaging device. For example, the system 200 may further comprise a medical imaging device in electrical communication with the control unit 215. The medical imaging device may capture one or more anatomical images of the patient and transmit the anatomical images to the control unit 215. In some embodiments, the anatomical image may be captured at the time of planning (e.g., as an initial step in performing the method 300) and/or in substantially real-time. In some embodiments, the anatomical image is received from a database or storage device. For example, anatomical images may be captured by a medical imaging device at any time prior to the IRE procedure and stored for later use. During planning, the anatomical images may be received from the database or the storage device for carrying out the method 300.

In some embodiments, the anatomical image of the patient includes a target tissue. The target tissue may comprise at least a portion of tissue to be ablated through the IRE procedure. For example, the target tissue may be a tumor or another diseased tissue to be treated by killing the cells through IRE. As shown in FIG. 4, a target tissue 410, such as a tumor, may be included in the anatomical image 405. In some embodiments, the target tissue may be more broadly defined to include a portion of tissue to be ablated as well as surrounding tissue. For example, it may be difficult to determine the exact boundaries of a diseased tissue, and thus the target tissue may be overinclusive to include some surrounding healthy tissue to ensure that the entire diseased tissue is included in the target tissue. In some embodiments, the target tissue includes only a portion of a diseased tissue.

The predicted target zone may also be referred to herein as a proposed target zone. The predicted target zone may comprise the target tissue. In some embodiments, the predicted target zone comprises the entire target tissue. In some embodiments, the predicted target zone may be more broadly defined to include more than just the target tissue. For example, it may be difficult to determine the exact boundaries of the target tissue and thus the predicted target zone may be overinclusive to include some surrounding tissue to ensure that the entire target tissue is included in the predicted target zone. In some embodiments, the predicted target zone includes only a portion of the target tissue.

In some embodiments, the predicted target zone is identified 310 based on the anatomical image. For example, the target tissue may be identified on the anatomical image, and the predicted target zone may be defined to include the target tissue. In some embodiments, the predicted target zone may be identified automatically by the control unit 215. In some embodiments, the control unit 215 may identify a target tissue and determine the predicted target zone based on the identified target tissue according to one or more rules. For example, the predicted target zone may encompass the entire target tissue. In another example, the predicted target zone may include a margin (i.e., a predetermined distance) beyond the target tissue.

In some embodiments, one or more image processing techniques as would be known to a person having an ordinary level of skill in the art may be utilized to identify a target tissue. For example, a tumor or other diseased tissue may be distinct from other tissue and surrounding structures on an anatomical image and may thus be identified by image processing.

In some embodiments, a machine learning algorithm may be utilized to identify the target tissue. For example, a machine learning algorithm may be trained using a set of training data. In some embodiments, the set of training data comprises synthetic anatomical images (i.e., not from real patients). In some embodiments, the set of training data comprises anatomical images from historical patients. The training data may be used to train the machine learning algorithm to identify tumors, lesions, or other diseased tissues. Accordingly, the machine learning algorithm may become more proficient in identifying the target tissue over time. Thus, a trained machine learning algorithm may identify the target tissue, thereby allowing the control unit 215 to identify 310 the predicted target zone based on the determined target tissue without human input.

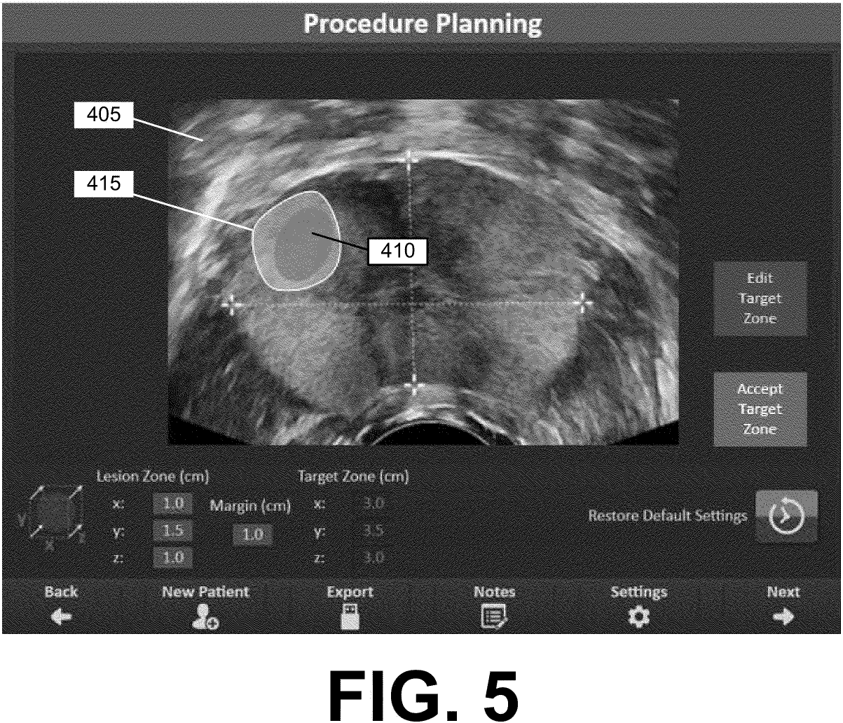

In some embodiments, displaying 315 the predicted target zone on the display device 205 comprises displaying the predicted target zone superimposed over the anatomical image. For example, FIG. 5 depicts an exemplary user interface stage for displaying a predicted target zone in accordance with an embodiment. As shown, a predicted target zone 415 is displayed 315 as an object superimposed on an anatomical image 405. In some embodiments, the predicted target zone 415 may encompass the target tissue 410 and thus may be superimposed over the target tissue 410. Accordingly, a user may view the predicted target zone 415 on the display device 205 to assess the predicted target zone 415 prepared by the control unit 215. While an exemplary manner of displaying 315 the predicted target zone is depicted herein, it should be understood that the predicted target zone may be displayed 315 on the user interface in variety of manners.

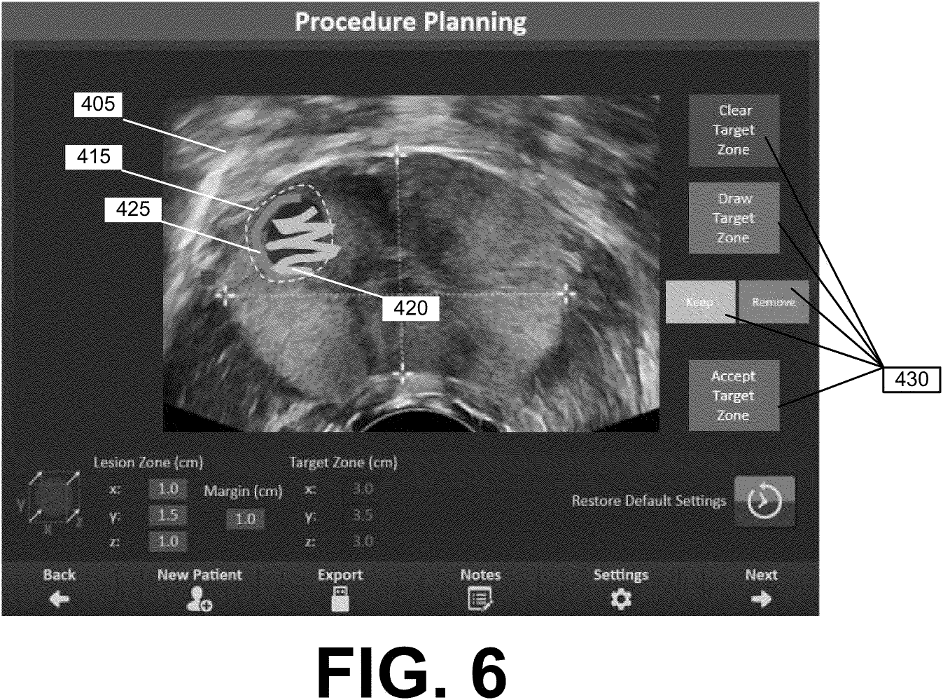

Turning now to FIG. 6, an exemplary user interface stage for obtaining target feedback related to a predicted target zone is depicted in accordance with an embodiment. In some embodiments, the feedback related to the predicted target zone (i.e., target feedback) may be an indication of portions of the predicted target zone to be retained. For example, as shown in FIG. 6, the user may mark one or more portions 420 to be retained from the predicted target zone 415 on the anatomical image 405. In some embodiments, the feedback related to the predicted target zone may be an indication of portions of the predicted target zone to be removed. For example, as shown in FIG. 6, the user may mark one or more portions 425 to be removed from the predicted target zone 415 on the anatomical image 405. In some embodiments, the feedback related to the predicted target zone may be an indication of approval or acceptance of the predicted target zone. It should be understood that the target feedback may comprise a combination of the described types of feedback. For example, the user may mark one or more portions 420 for retention, mark one or more portion 425 for removal, and then accept the predicted target zone with the indicated changes.

In some embodiments, the feedback related to the predicted target zone may comprise additional types of feedback. In some embodiments, the target feedback may be an indication to clear the entire predicted target zone. For example, if a user is unsatisfied with the predicted target zone, the user may choose to clear the predicted target zone from the user interface. In some embodiments, the target feedback may be an indication to select a new target zone. For example, a user may desire to select or draw a new target zone separate from the predicted target zone (e.g., if the user has chosen to clear the predicted target zone and start over).

In some embodiments, the feedback related to the predicted target zone may be received 320 via the at least one input device 210. As shown in FIG. 6, the user interface may display one or more buttons or icons 430 for selecting different types of target feedback and entering the feedback. For example, a user may use an input device (e.g., a mouse, a trackpad, or a touchscreen) to select a function from the icons 430. In some instances, some functions as described may require additional action. For example, if the user selects the icon 430 to indicate portions 420 of the predicted target zone 415 for retention, the user may then use the input device 210 to select the portions 420 on the anatomical image 405. In another example, if the user selects the icon 430 to indicate portions 425 of the predicted target zone 415 for removal, the user may then use the input device 210 to select the portions 425 on the anatomical image 405. In another example, if the user selects the icon 430 to select a new target zone, the user may then use the input device 210 to draw the target zone on the anatomical image 405.

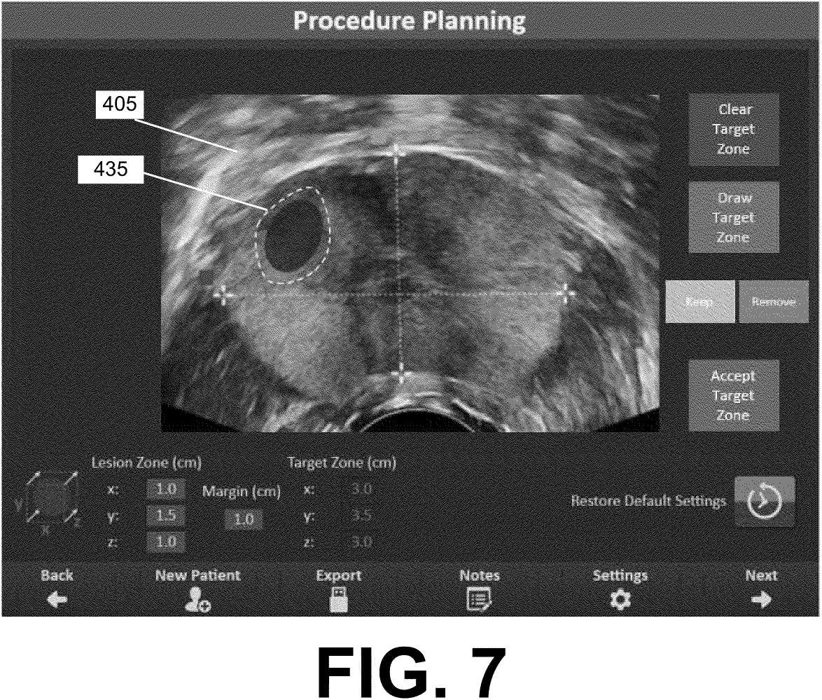

In some embodiments, modifying 325 the predicted target zone based on the feedback to define a selected target zone comprises incorporating any changes indicated by the user as target feedback. FIG. 7 depicts an exemplary user interface stage comprising a selected target zone obtained by modifying the predicted target zone in accordance with an embodiment. As shown in FIG. 7, the selected target zone 435 may be different from the predicted target zone 415. For example, the predicted target zone 415 may be modified to retain portions 420 marked for retention to obtain the selected target zone 435. In some embodiments, all portions not marked for retention are removed. In another example, the predicted target zone 415 may be modified to remove portions 425 marked for removal to obtain the selected target zone 435. In some embodiments, all portions not marked for removal are retained. In another example, a new target zone drawn by the user on the anatomical image 405 may replace the predicted target zone 415 such that the selected target zone 435 substantially comprises the new target zone drawn by the user. It should be understood that the selected target zone 435 may be obtained by multiple types of modifications as described herein. In some embodiments, the user may perform multiple cycles, i.e., multiple iterations, of modifying the predicted target zone and accepting or approving a target zone prior to finalizing the selected target zone 435.

In some embodiments, the ablation plan generated 330 based on the selected target zone 435 may comprise the selected target zone 435. As such, the ablation plan may comprise a set of steps and conditions that result in ablating the selected target zone. In some embodiments, the ablation plan may comprise additional plan information for carrying out the IRE procedure.

In some embodiments, the control unit 215 may measure one or more distances in the anatomical image 405 to determine dimensions of the selected target zone 435. In some embodiments, the control unit 215 may define a location of the selected target zone based on the anatomical image. In some embodiments, the ablation plan may contain the one or more distances, the one or more dimensions, and/or the location associated with the selected target zone. For example, several measurements of a lesion zone (i.e., a target tissue), a selected target zone, and/or a margin zone as described herein may be displayed on a user interface. Furthermore, the control unit 215 may measure additional distances in the anatomical image related to regions other than the selected target zone 435. For example, the control unit 215 may measure distances to surrounding tissue, e.g., healthy tissue and/or delicate surrounding tissue structures for which ablation may be avoided. In some embodiments, the user may modify one or more of the values for the measurements. In some embodiments, various parameters of the IRE procedure may be determined based on the ablation plan information as would be apparent to a person having an ordinary level of skill in the art.

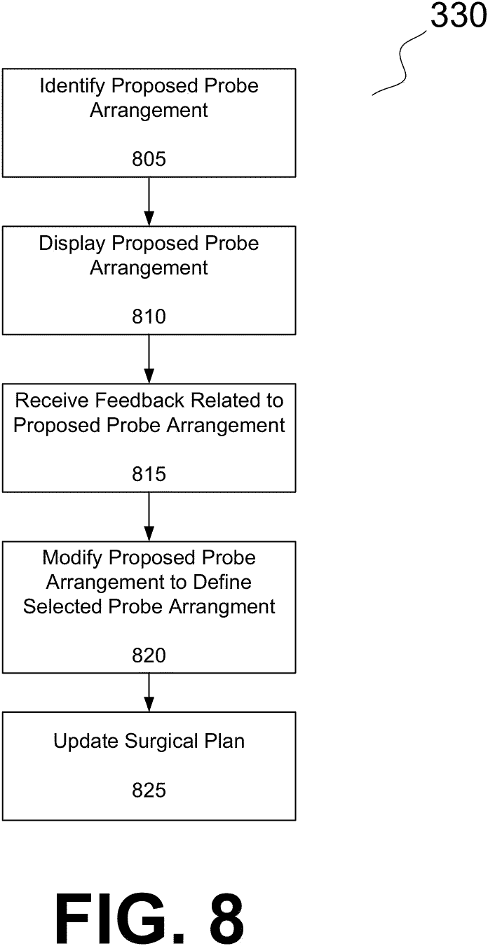

In some embodiments, the ablation plan generated by the control unit 215 comprises a probe arrangement for carrying out the IRE procedure. Turning now to FIG. 8, a flow diagram of an illustrative computer-implemented method for generating 330 an ablation plan comprising a probe arrangement by the system 200 is depicted in accordance with an embodiment. For example, the method 800 may be carried out by the processor of the control unit 215 upon execution of the instructions stored on the memory. It should be understood that in some embodiments, generating 330 an ablation plan in the method 300 may comprise, among other things, performing the method 800. The method 800 comprises identifying 805 a proposed probe arrangement, displaying 810 the proposed probe arrangement on the display device 205, receiving 815 feedback related to the proposed probe arrangement, modifying 820 the proposed probe arrangement based on the feedback to define a selected probe arrangement, and updating 825 the ablation plan based on the selected probe arrangement.

In some embodiments, the proposed probe arrangement is identified based on the selected target zone. In some embodiments, the proposed probe arrangement comprises a recommended number of probes (e.g., one, two, three, four, five, six, or more probes). Furthermore, the probes probe arrangement may comprise a position for each of a plurality of probes based on the recommended number of probes. In some embodiments, the measured distances, dimensions, and/or locations associated with the selected target zone may be used to determine the proposed probe arrangement. As would be understood by a person having an ordinary level of skill in the art, the proposed probe arrangement may be designed to provide adequate ablation across the entirety of the selected target zone. In some embodiments, adequate ablation is defined by an electric field threshold (EFT), i.e., a minimum electric field strength created at a particular location. In some embodiments, the EFT may be set at a value that is lethal for the tissue such that the entirety of the selected target zone is exposed to a lethal dose of electrical stimulation according to the ablation plan. Furthermore, the EFT may be increased above a lethal dose in order to provide a margin of error and ensure adequate ablation across the selected target zone. In some embodiments, the EFT is about 700 V/cm. In some embodiments, the EFT is about 800 V/cm. In some embodiments, the EFT is about 900 V/cm. In some embodiments, the EFT is about 1000 V/cm. In some embodiments, the EFT is about 1500 V/cm. In some embodiments, the EFT is about 2000 V/cm. In some embodiments, the EFT is greater than about 2000 V/cm. However, additional values may be used for the EFT based on the type of target tissue and the conditions of the IRE procedure as would be known to a person having an ordinary level of skill in the art including values within ranges between any of the values listed above, inclusive of such values. In some embodiments, the user may indicate the type of tissue being treated using the input device 210, and the EFT used to set the proposed probe arrangement may be adjusted based on the indicated type of tissue. In some embodiments, the control unit 215 may determine the type of tissue being treated using a machine learning algorithm as described herein, and the EFT used to set the proposed probe arrangement may be adjusted based on the determined type of tissue. Accordingly, the proposed probe arrangement including a position for each probe of a plurality of probes may be chosen to satisfy the EFT across the entirety of the selected target zone.

In some embodiments, the proposed probe arrangement may also be constrained by a maximum electric field strength, i.e., the probe arrangement should not result in an electric field strength above a predetermined maximum at any location across the selected target zone for safety purposes.

In some embodiments, a machine learning algorithm may be utilized to identify 805 a proposed probe arrangement. For example, a machine learning algorithm may be trained using a set of training data. In some embodiments, the set of training data comprises synthetic anatomical images and ablation plan data (i.e., not from real patients). In some embodiments, the set of training data comprises anatomical images and ablation plan data from historical patients. The training data may be used to train the machine learning algorithm to identify probe arrangements that satisfy the electric field conditions to provide effective ablation within a margin of safety. For example, the training data may include outcomes from ablation plans that were implemented on historical patients such that there is an indication of success or failure, and the machine learning algorithm may be trained to select successful probe arrangements for a given size, shape, and/or tissue type of a selected target zone 435. Accordingly, the machine learning algorithm may become more proficient in identifying proposed probe arrangements over time. Thus, a trained machine learning algorithm may enable the control unit 215 to identify the proposed probe arrangement based on the selected target zone 435 without human input.

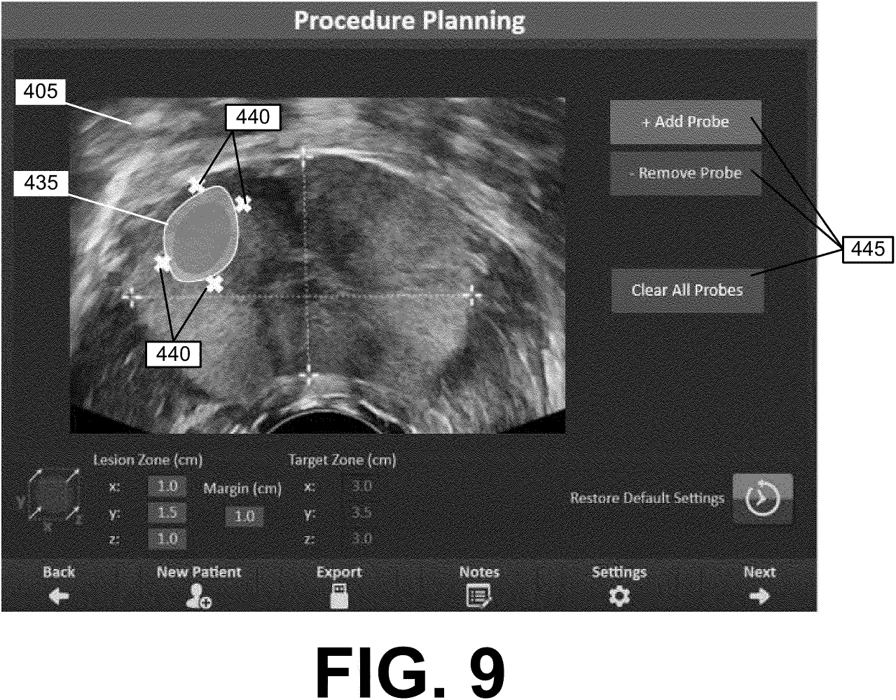

In some embodiments, displaying 810 the proposed probe arrangement on the display device 205 comprises displaying the proposed probe arrangement superimposed over the anatomical image. For example, FIG. 9 depicts an exemplary user interface stage for planning a probe arrangement in accordance with an embodiment. As shown, a marked position 440 may be displayed superimposed over the anatomical image 405 for each probe of a plurality of probes in the proposed probe arrangement. In some embodiments, the selected target zone 435 may also be displayed superimposed over the anatomical image 405 such that the proposed probe arrangement may also be adjacent and/or superimposed over the selected target zone 435. Accordingly, a user may view the proposed probe arrangement on the display device 205 to assess the marked position 440 for each probe. While an exemplary manner of displaying 810 the proposed probe arrangement is depicted herein, it should be understood that the proposed probe arrangement may be displayed 810 on the user interface in variety of manners.

In some embodiments, the feedback related to the proposed probe arrangement (i.e., probe feedback) may be an indication of one or more of the plurality of probes to be removed from the proposed probe arrangement. For example, the user may select a marked position 440 of a probe on the user interface and indicate its removal from the proposed probe arrangement. In another example, the user may indicate to remove a probe from the proposed probe arrangement without selecting a specific probe. Accordingly, a probe may be removed from the proposed probe arrangement and the marked positions 440 for the remaining probes of the plurality of probes may be automatically adjusted. In some embodiments, the feedback related to the proposed probe arrangement (i.e., probe feedback) may be an indication to add one or more additional probes to the plurality of probes in the proposed probe arrangement. For example, the user may indicate the addition of a probe, and the user interface may display an additional probe (i.e., an additional marked position 440 for a new probe in the proposed probe arrangement). In some embodiments, the feedback related to the proposed probe arrangement may be an indication to adjust a marked position 440 of a probe of the plurality of probes. For example, the user may select a marked position 440 of a probe, and re-position the marked position 440 on the anatomical image 405. In some embodiments, the feedback related to the proposed probe arrangement may be an indication of approval or acceptance of the proposed probe arrangement. It should be understood that the probe feedback may comprise a combination of the described types of feedback. For example, the user may add or remove one or more probes from the proposed probe arrangement, adjust a marked position 440 for one or more probes, and accept the proposed probe arrangement with the indicated changes.

In some embodiments, the feedback related to the proposed probe arrangement may be received 815 via the at least one input device 210. As shown in FIG. 9, the user interface may display one or more buttons or icons 445 for selecting different types of target feedback and entering the feedback. For example, a user may use an input device (e.g., a mouse, a trackpad, or a touchscreen) to select a function from the icons 445. In some instances, some functions as described may require additional action. For example, if the user selects the icon 445 to remove a probe from the proposed probe arrangement, the user may also use the input device 210 to select the probe to be removed from the proposed probe arrangement. In another example, if the user selects the icon 445 to add a probe to the proposed probe arrangement, the user may also use the input device 210 to select a marked position 440 for the new probe. In some instances, the new probe may be automatically positioned and/or the marked positions 440 for additional probes of the plurality of probes may be automatically adjusted when the new probe is added.

In some embodiments, the feedback related to the proposed probe arrangement may comprise additional types of feedback. In some embodiments, the probe feedback may be an indication to clear the entire proposed probe arrangement. For example, if a user is unsatisfied with the proposed probe arrangement, the user may choose to clear the proposed probe arrangement from the user interface and/or to add and place probes manually.

In some embodiments, modifying 820 the proposed probe arrangement based on the feedback to define a selected probe arrangement comprises incorporating any changes indicated by the user as probe feedback. For example, the proposed probe arrangement may be modified to remove probes, add probes, and/or re-position the marked positions 440 for probes as indicated to obtain the selected probe arrangement. In examples where probes are added or removed, the marked positions 440 of the plurality of probes may be automatically adjusted to account for the modified number of probes in the proposed probe arrangement. In another example, a new probe arrangement made by the user on the anatomical image 405 may replace the proposed probe arrangement such that the selected probe arrangement substantially comprises the new probe arrangement made by the user. It should be understood that the selected probe arrangement may be obtained by multiple types of modifications as described herein. In some embodiments, the user may perform multiple cycles, i.e., multiple iterations, of modifying the proposed probe arrangement and accepting or approving prior to finalizing the selected probe arrangement.

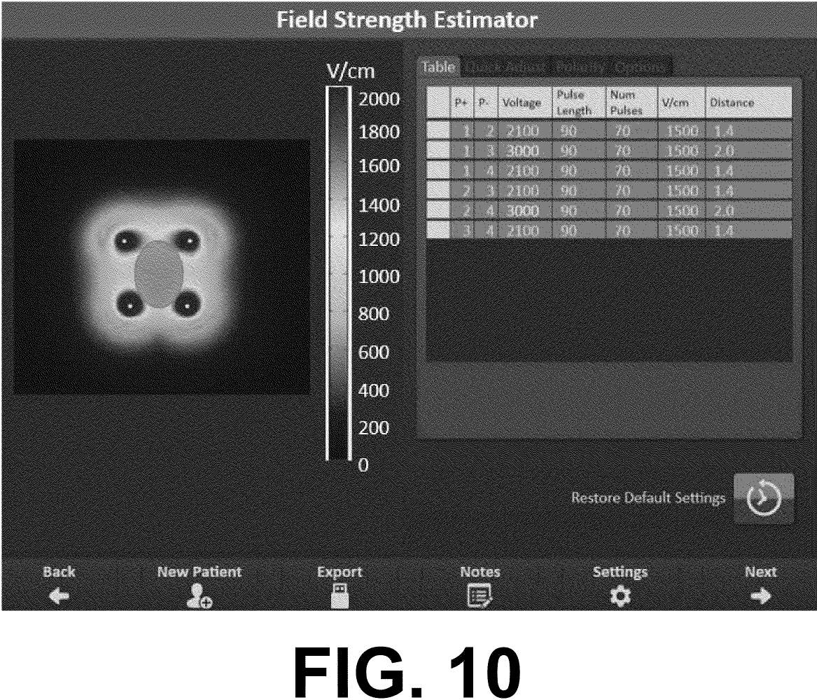

In some embodiments, updating 825 the ablation plan based on the selected probe arrangement comprises incorporating the selected probe arrangement into the ablation plan. In some embodiments, the ablation plan comprises a planned series of pulses to be emitted between the plurality of probes. The planned series of pulses may include an ordered set of pulses, each pulse being emitted between a specific pair of probes, according to various treatment parameters as described herein. Accordingly, the ablation plan may be updated 825 to adjust the planned series of pulses based on the selected probe arrangement. In some embodiments, once the probe arrangement has been changed as described herein, the planned series of pulses may be adjusted in order to satisfy the EFT across the entirety of the selected target zone and/or to ensure that the electric field strength does not exceed the predetermined maximum at any point across the selected target zone. In some embodiments, once the probe arrangement has been changed as described herein, one or more treatment parameters for any number of pulses of the planned series of pulses may be adjusted in order to satisfy the EFT across the entirety of the selected target zone and/or to ensure that electric field strength does not exceed the predetermined maximum at any point across the selected target zone. In some embodiments, the treatment parameters comprise one or more of a voltage, a pulse length, a number of pulses, and a pulse path. In some embodiments, a map of electric field strength across the selected target zone may be displayed to the user on the user interface via the display device 205. For example, FIG. 10 depicts an exemplary user interface stage comprising an electric field strength map in accordance with an embodiment. In some embodiments, pulse information, e.g., treatment parameters and/or information related to the planned series of pulses, may be displayed to the user as shown. In some embodiments, the user may modify pulse information using the input device 210.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

In some embodiments, the machine learning algorithms for identifying 310 a predicted target zone and/or identifying 805 a proposed probe arrangement may be re-trained over time and thus improved. For example, the machine learning algorithm may be trained using a first set of training data, which may be “seed data.” The seed data may comprise synthetic images and/or historical patient images and related ablation plan data and/or outcome data. The seed data may be of at least a critical volume to enable the machine learning algorithm to satisfactorily identify 310 predicted target zones and/or identify 805 proposed probe arrangements in live patients. Following the performance of the method 300, the anatomical images of the patient, the ablation plan data associated therewith, and the outcome data associated therewith may be used to further train the machine learning algorithms. For example, where a particular predicted target zone or proposed probe arrangement is rejected by the user (e.g., heavily modified and/or entirely cleared), the machine learning algorithm may obtain an indication of these outcomes and may be trained over time to provide different and/or better predictions or proposals in similar scenarios. In another example, where a particular predicted target zone or proposed probe arrangement received positive feedback from the user (e.g., substantially and/or entirely accepted as presented), the machine learning algorithm may obtain an indication of these outcomes and may be trained over time to provide similar predictions or proposals in similar scenarios. In another example, after an IRE procedure is performed, the machine learning algorithm may obtain outcome data associated with the success or failure of the procedure and may be trained over time to recognize ablation plans with a high likelihood of success and/or a low likelihood of success. Accordingly, live cases may be used to form a second set of training data, which may be “refinement data” that is used on a continual basis to re-train the machine learning algorithms.

In some embodiments, the control unit 215 and/or the machine learning algorithms employed by the system 200 are configured to detect additional tissues or structures in addition to the target tissue. In some embodiments, the control unit 215 is configured to identify interfering structures in the anatomical images of the patient, e.g., blood vessels, bones, nerves, organs, and/or other delicate biological structures as would be known to a person having an ordinary level of skill in the art. Accordingly, the suggestions presented by the control unit 215 including the predicted target zone and the proposed probe arrangement may account for the interfering structures. For example, the predicted target zone may be chosen to avoid an interfering structure. In another example, the proposed probe arrangement may be chosen to avoid placing a probe at a location that would be difficult or impractical due to the presence of an interfering structure. In another example, the proposed probe arrangement may be chosen to prevent application of electrical pulses to an interfering structure at a strength that would result in ablation of the interfering structure. In order to enable such determinations for planning, the control unit 215 may measure various parameters relating to such interfering structures, e.g., distances or dimensions of interfering structures and/or distances or dimensions between the interfering structures and the target tissues. Accordingly, the ablation plan including the predicted target zone, the planned probe arrangement, the series of planned pulses, and/or the treatment parameters may be adjusted based on knowledge or identification of interfering structures in the anatomical images.

While the systems and methods described herein are generally discussed as using individual probes, it should be understood that multi-electrode devices (e.g., a SID) may be used herein with appropriate modifications as would be apparent to a person having an ordinary level of skill in the art. For example, the proposed probe arrangement may account for the type of probe device being utilized and the ability of the user to add, remove, or re-position probes may be constrained by the type of probe device being utilized.

While the systems and methods described herein generally related to IRE procedures, it is to be understood that the present disclosure is equally applicable to other non-thermal ablation techniques, such as, reversible electroporation, electro-chemotherapy (ECT), high frequency irreversible electroporation (HFIRE), pulsed field ablation (PFA), pulsed electric fields (PEF), and the like as would be apparent to a person having an ordinary level of skill in the art.

Additional details and description of systems and methods for planning IRE and/or other electroporation-based therapies are disclosed in U.S. Pat. Application No. 12/906,923, now issued as U.S. Pat. No. 9,198,733, each of which is incorporated herein by reference in its entirety.

### Systems for Preparing an IRE Procedure

As discussed herein, IRE procedures are particularly useful in scenarios requiring precision and conservation of the extracellular matrix, blood flow, nerves, and other tissue. Accordingly, once an ablation plan has been determined, ensuring compliance with the ablation plan is critical to ensure that the target tissue is fully destroyed and that delicate structures surrounding the target tissue are preserved. However, accurately placing electrodes in the correct position and orientation is a complex and time-consuming process. As such, it would be advantageous to have a tool for facilitating guided placement of IRE electrodes with greater accuracy and reduced preparation time.

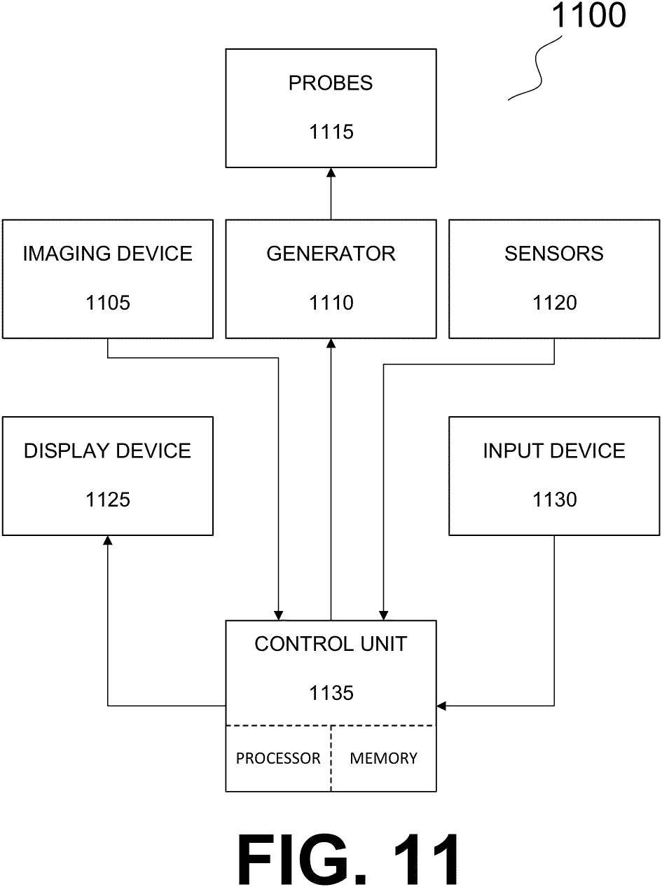

Turning now to FIG. 11, a block diagram of an illustrative system for placing electrodes for an IRE procedure is depicted in accordance with an embodiment. The system 1100 comprises an imaging device 1105, a generator 1110, a plurality of probes 1115 in electrical communication with the generator 1110, one or more sensors 1120, a display device 1125, at least one input device 1130, and a control unit 1135 comprising a processor and a memory. The imaging device 1105, the generator 1110, the sensors 1120, the display device 1125, and the at least one input device 1130 are each in electrical communication with the control unit 1135.

The imaging device 1105 may be configured to collect an anatomical image of a patient, e.g., an anatomical image comprising a target tissue as described herein. In some embodiments, the imaging device 1105 is an ultrasound imaging device. In some embodiments, the imaging device 1105 is a CT imaging device. In some embodiments, the imaging device 1105 is a PET imaging device. In some embodiments, the imaging device 1105 is an x-ray imaging device. However, it should be understood that any type of imaging device 1105 appropriate for identifying a target tissue may be utilized herein as would be known to a person having an ordinary level of skill in the art.

In some embodiments, the imaging device 1105 is configured to capture one or more anatomical images of the patient and transmit the anatomical images to the control unit 1135. In some embodiments, the anatomical images may be captured in substantially real-time.

In some embodiments, the generator 1110 is configured to generate one or more electrical pulses for delivery to a target tissue. In some embodiments, the generator 1110 is configured to emit the electrical pulses through the plurality of probes 1115 to the target tissue. In some embodiments, the generator 1110 is configured to be controlled by the control unit 1135, i.e., the generator 1110 responds to signals from the control unit 1135 to generate electrical pulses, e.g., according to an ablation plan. It should be understood the generator 1110 may be a generator of an IRE system as described herein with respect to FIG. 1 and may comprise any of the features and/or functions as described herein.

In some embodiments, the plurality of probes 1115 comprises two probes. In some embodiments, the plurality of probes 1115 comprises three probes. In some embodiments, the plurality of probes 1115 comprises four probes. In some embodiments, the plurality of probes 1115 comprises five probes. In some embodiments, the plurality of probes 1115 comprises six probes. In some embodiments, the plurality of probes 1115 comprises more than six probes. However, any number of probes may be utilized herein as would be known to a person having an ordinary level of skill in the art.

In some embodiments, the plurality of probes 1115 comprises separate probes 1115 that may be individually placed. In some embodiments, the plurality of probes 1115 comprises one or more multi-electrode probes, e.g., a SID including two electrodes spaced apart on a single probe.

In some embodiments, the sensors 1120 may be configured to indicate a position of each probe of the plurality of probes 1115. In some embodiments, each probe comprises a sensor 1120 configured to indicate a position of the probe. In some embodiments, a single sensor 1120 may indicate the position of multiple probes. In some embodiments, a plurality of sensors 1120 may indicate the position of a single probe. In some embodiments, the sensors 1120 are coupled to the plurality of probes 1115. In some embodiments, the sensors 1120 are remotely located from the plurality of probes 1115.

In a particular embodiment, the sensors 1120 are electromagnetic (EM) sensors. For example, the sensors 1120 may be EM coils located on the each of the plurality of probes 1115. The system 1100 may further comprise an EM generator configured to generate an EM field within the operating space. In some embodiments, the EM generator is placed above or below the patient in the operation environment. However, the EM generator may be placed in any location that enables generation of an EM field in a region surrounding the target tissue. The sensors 1120 may be EM sensors configured to detect the EM field, which may vary throughout the operation environment. The sensors 1120 may provide a signal indicating the EM field characteristics (e.g., a magnitude and/or a direction) detected at a particular location to the control unit 1135, and the control unit 1135 may use the detected EM field characteristics to determine a location of the sensor 1120 and thus a location of the corresponding probe of the plurality of probes 1115. In some embodiments, the control unit 1135 may utilize a predetermined geometry between the sensor 1120 and the corresponding probe to determine the location of a probe tip. In some embodiments, the sensors 1120 enable determination of the position of the plurality of probes 1115 in six degrees of freedom, i.e., determination of location and orientation. While it may be possible to determine location and orientation with a single sensor 1120, in some embodiments a probe may comprise multiple sensors 1120 (e.g., one sensor 1120 at the probe tip and one sensor 1120 elsewhere on the probe body) to enable determination of all six degrees of freedom of the probe position.

In some embodiments, the sensors 1120 may be co-registered with the imaging device 1105. For example, the sensors 1120 and/or the imaging device 1105 may undergo a registration or calibration procedure to determine a location of the sensors 1120 with respect to the outputted anatomical image view from the imaging device 1105. Accordingly, the co-registration may enable tracking of the plurality of probes 1115 with respect to anatomy of the patient, e.g., the target tissue.

The display device 1125 may be a visual display configured to render visual information, which may include any graphical or textual information as described herein. For example, the display device 1125 may present a user interface to a user that includes graphical or textual depictions of information related to an IRE procedure. As further described herein, the user interface may include several stages, i.e., different panels that are presented at different steps of probe placement and/or execution of IRE. In some embodiments, the display device 1125 is a touchscreen or a touch-sensitive display. In some embodiments, the display device 1125 is wireless display remotely located from other components of the system 1100. It should be understood the display device 1125 may be a display, such as display 104 as described herein with respect to FIG. 1 and/or display 205 as described herein with respect to FIG. 2, and may comprise any of the features and/or functions as described with respect to the display 104 and/or display 205.

The at least one input device 1130 may comprise any number and type of input devices configured to receive and/or process input from a user. For example, the input device 1130 may include a mouse, a trackpad, a keyboard, a touchscreen, a microphone, a foot pedal, one or more knobs, one or more sliders, one or more switches, one or more user interaction components, and the like. However, any type of input device 1130 as would be known to a person having an ordinary level of skill in the art may be utilized herein. It should be understood the input device 1130 may be an input device 106 as described herein with respect to FIG. 1 and/or input device 210 as described herein with respect to FIG. 2 and may comprise any of the features and/or functions as described with respect to the input devices 106 and/or input devices 210.

The control unit 1135 may be in electrical communication with the imaging device 1105, the generator 1110, the sensors 1120, the display device 1125, and the input device 1130 in order to enable a user to prepare for an IRE procedure and/or execute the IRE procedure according to an ablation plan. In some embodiments, the control unit 1135 includes a processor and a memory such as a non-transitory, computer-readable medium storing instructions for presenting a user interface and placing probes for an IRE procedure. It should be understood that the control unit 1135 may comprise any number of components of the processor 108, the memory 110, and/or the user interface system 100 as described herein with respect to FIG. 1 (e.g., the communications interface 102 and/or the one or more output devices 112) and may comprise any of the features and/or functions as described with respect to the user interface system 100. It should also be understood that the control unit 1135 may comprise any number of components of the control unit 215 as described herein with respect to FIG. 2 and may comprise any of the features and/or functions as described with respect to the system 200.

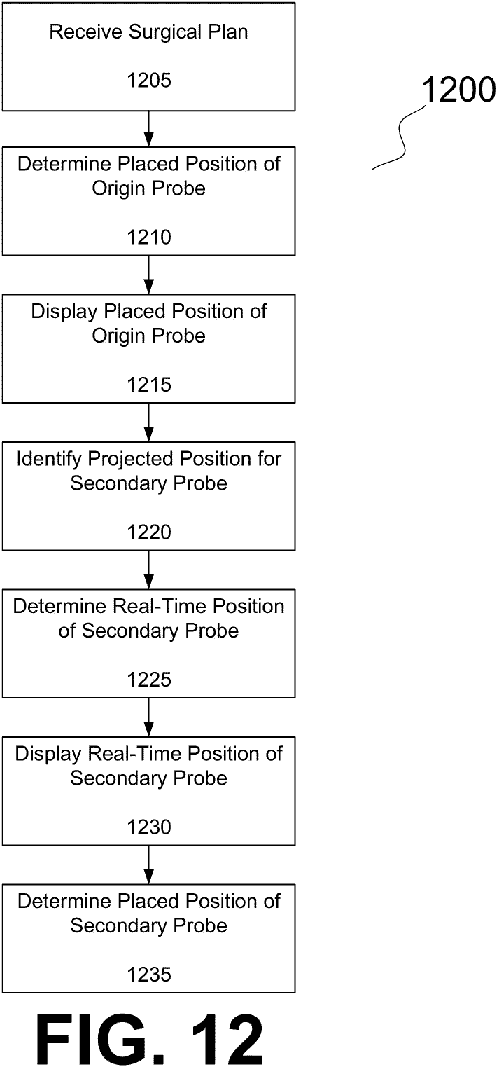

Turning now to FIG. 12, a flow diagram of an illustrative computer-implemented method for preparing an IRE procedure by the system 1100 is depicted in accordance with an embodiment. For example, the method 1200 may be carried out by the processor of the control unit 1135 upon execution of the instructions stored on the memory. The method 1200 comprises receiving 1205 an ablation plan including a planned probe arrangement, determining 1210 a placed position of an origin probe of the plurality of probes 1115, displaying 1215 the placed position of the origin probe on the display device 1125, identifying 1220 a projected position for a secondary probe of the plurality of probes 1115, determining 1225 a real-time position of the secondary probe, displaying 1230 the projected position and the real-time position for the secondary probe on the display device 1125, and determining 1235 a placed position of the secondary probe.

In some embodiments, the ablation plan comprises a planned probe arrangement and target information related to a target zone, e.g., a target tissue. In some embodiments, the target information includes a shape, size, dimensions, and/or location of a target zone. In some embodiments, the planned probe arrangement includes a planned position for each of the plurality of probes 1115. In some embodiments, the planned positions are relative to the remaining probes 1115. In some embodiments, the planned positions are relative to the target zone and/or the target tissue of the patient.

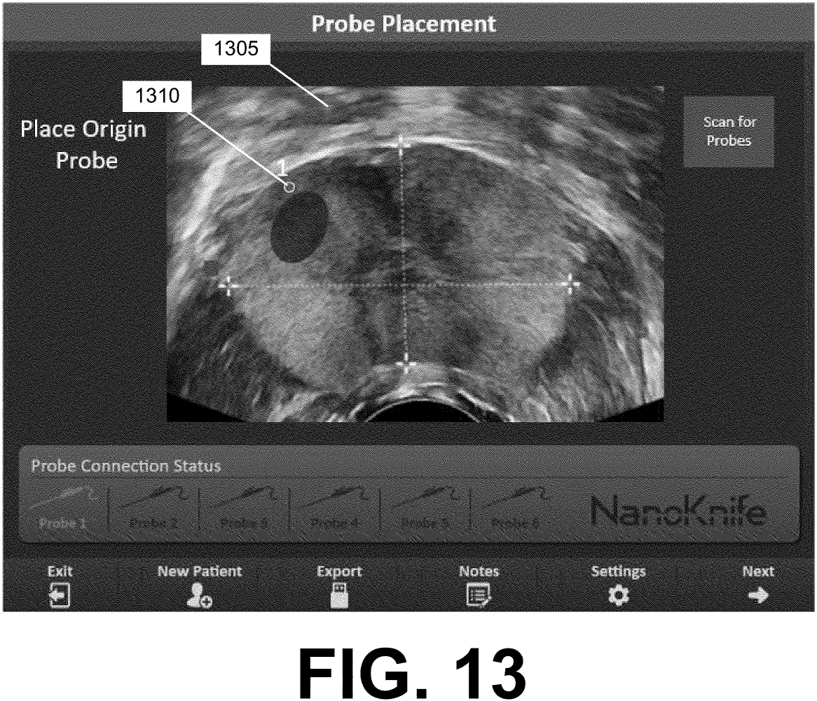

In some embodiments, the placed position of the origin probe is determined 1210 based on signals from the sensor(s) 1120 corresponding to the origin probe. Furthermore, because the sensors 1120 are co-registered to the output of the imaging device 1105, the placed position is known with respect to the anatomical image of the patient. In some embodiments, the placed position of the origin probe is displayed 1215 on the display device 1125 superimposed over the anatomical image. For example, FIG. 13 depicts an exemplary user interface stage for displaying a placed position of an origin probe in accordance with an embodiment. As shown, an anatomical image 1305 of the patient and a placed position 1310 of the origin probe may be displayed in superimposed fashion. In some embodiments, the user provides input via the input device 1130 to initiate scanning for the probes 1115. Accordingly, the user may be able to view the placed position of the origin probe with respect to the anatomy of the patient.

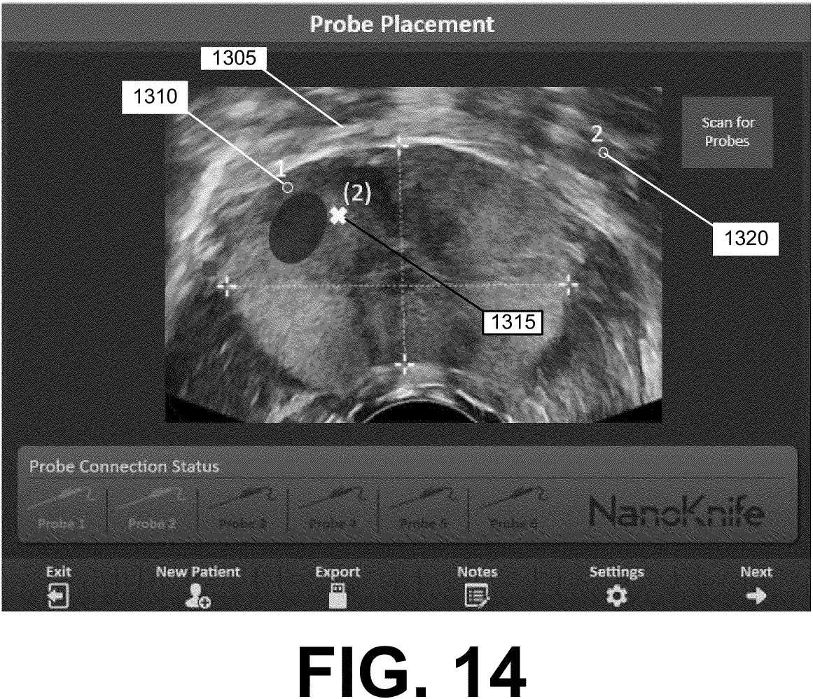

In some embodiments, the projected position for the secondary probe is identified 1220 based on the placed position of the origin probe and the planned probe arrangement. The planned probe arrangement may include relative locations and/or distances between the plurality of probes according to the ablation plan. Thus, using the placed position of the origin probe as a reference point, the projected position for the secondary probe may be identified 1220. For example, FIG. 14 depicts an exemplary user interface stage for guiding placement of a secondary probe in accordance with an embodiment. As shown, a projected position 1315 of the secondary probe may be marked with respect to the placed position 1310 of the origin probe on the anatomical image 1305. It should be understood that the projected position for all other probes of the plurality of probes 1115 may be determined in the same manner.

In some embodiments, the real-time position of the secondary probe is determined 1225 based on signals from the sensor(s) 1120 corresponding to the secondary probe. Furthermore, because the sensors 1120 are co-registered to the output of the imaging device 1105, the real-time position of the secondary probe is known with respect to the anatomical image of the patient. In some embodiments, the projected position and the real-time position of the secondary probe is displayed 1230 on the display device 1125 superimposed over the anatomical image. Accordingly, the user may be able to view the real-time position of the secondary probe with respect to the anatomy of the patient and guide the secondary probe towards the projected position. As shown in FIG. 14, a real-time position 1320 of the secondary probe may be displayed 1230 with respect to the projected position 1315 of the secondary probe on the anatomical image 1305 such that the user may move the secondary probe to place it at or near the projected position 1315. It should be understood that the real-time position for all other probes of the plurality of probes 1115 may be viewed in the same manner during placement thereof.

In some embodiments, the placed position of the secondary probe is determined 1235 based on the real-time position of the secondary probe. For example, the control unit 1135 may continuously monitor and update the real-time position of the secondary probe until placement is completed. In some embodiments, the control unit 1135 may receive input from a user via the input device 1130 to indicate that placement of the secondary probe is complete. In some embodiments, the control unit 1135 may determine that placement is complete when movement of the secondary probe ceases under certain conditions. For example, cessation of movement of the secondary probe within a predetermined distance of the projected position for the secondary probe may indicate that placement is complete.

In some embodiments, the placed position of the secondary probe may be guided based on the real-time position of the secondary probe and the projected position for the secondary probe. For example, the real-time position of the secondary probe may be displayed 1230 on display device 1125 in a first color (e.g., blue). When the real-time position of the secondary probe is within a threshold distance of the projected position for the secondary probe, the first color may be changed to a second color (e.g., green) such that real-time position of the secondary probe may be displayed 1230 on display device 1125 in the second color, thereby indicating adequate placement of the secondary probe to the user. In some embodiments, more than two colors may be used to provide feedback to the user related to the placement of the secondary probe. For example, a first color (e.g., red) may indicate that the placement is poor such that the ablation plan cannot be feasibly carried out as planned even with substantial adjustments. A second color (e.g., blue) may indicate that the placement is adequate, e.g., such that the ablation plan can be feasibly carried out with moderate or substantial adjustments. A third color (e.g., green) may indicate that the placement is relatively good, e.g., such that the ablation plan may be carried out with minor, limited, and/ or substantially no adjustments. It should be understood that the colors for different states as described are merely exemplary and any combination of colors may be utilized to provide feedback to the user related to the placement of the secondary probe. Furthermore, in some embodiments, other types of visual feedback may be provided via the user interface as would be apparent to a person having an ordinary level of skill in the art. Additionally, the user may additionally or alternatively be provided feedback related to the placement of the secondary probe in other forms, e.g., auditory feedback, tactile feedback, and the like, as would be apparent to a person having an ordinary level of skill in the art.

In some embodiments, the ablation plan may be modified or updated based on the placed position of the secondary probe. In an ideal case, the placed position of the secondary probe coincides exactly with the projected position of the secondary probe. However, the placed position and the projected position may not match in some instances. Accordingly, the method 1200 may further comprise comparing the placed position of the secondary probe to the projected position of the secondary probe and modifying the ablation plan based on the comparison. In some embodiments, modifying the ablation plan comprises updating a projected position for one or more additional probes (e.g., tertiary probes) of the plurality of probes based on the comparison. The control unit 1135 may update the projected position for one or more probes to be subsequently placed in order to maintain the planned probe arrangement. In some instances, it may not be entirely possible to maintain the planned probe arrangement, so the control unit 1135 may attempt to minimize the deviation from the planned probe arrangement. In some embodiments, modifying the ablation plan comprises updating one or more treatment parameters of the ablation plan based on the comparison. In some embodiments, the treatment parameters comprise one or more of a voltage, a pulse length, a number of pulses, and a pulse path. The control unit 1135 may update the treatment parameters in order to maintain the electric field strength in one or more locations across the target zone. For example, the control unit 1135 may update the treatment parameters in order to exactly match a planned electric field strength map (e.g., as shown in FIG. 10) despite the displacement of the secondary probe from the projected position. In another example, the control unit 1135 may update the treatment parameters in order to minimize deviation from the planned electric field strength map of the ablation plan. In some embodiments, modifying the ablation plan comprises a combination of updating a projected position for one or more additional probes and updating one or more treatment parameters of the ablation plan.

In some embodiments, where the placed position and the projected position do not match, the user may be presented several options to proceed based on the placed position of the probe. For example, the system 1100 may prompt the user on the user interface to select between repositioning the secondary probe and updating the ablation plan to account for the placed position of the secondary probe. Accordingly, the user may select the option to reposition the secondary probe to match and/or improve the match with the projected position, thereby allowing the system 1100 to retain the ablation plan without substantial modifications. Alternatively, the user may selected the option to update the ablation plan based on the placed position of the secondary probe, thereby allowing the user to retain the secondary probe in its placed position.

It should be understood that the ablation plan may be modified based on placement of subsequent probes in the same manner. Accordingly, the ablation plan may be updated after placement of a third probe, a fourth probe, and so on until all probes are placed.

As discussed herein, a machine learning algorithm may be utilized to identify proposed probe arrangements for the plurality of probes. Accordingly, a machine learning algorithm may also be utilized to update the projected position for subsequent probes. The machine learning algorithm may comprise any of the features and/or functions of the machine learning algorithm used to identify proposed probe arrangements in the method 300. For example, the machine learning algorithm may be trained using a set of training data to identify probe arrangements that satisfy the electric field conditions to provide effective ablation within a margin of safety. For example, the machine learning algorithms may be intermittently re-trained and thus improved using refinement data including anatomical images of the patient, ablation plan data associated therewith, and outcome data associated therewith.

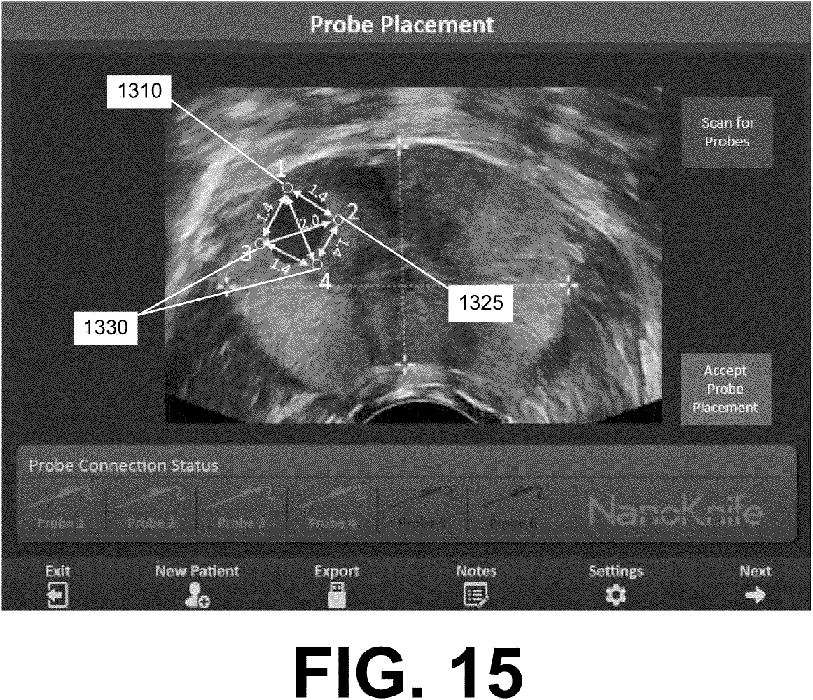

In some embodiments, after placement of each of the plurality of probes 1115, the control unit 1135 may measure one or more distances related to the probes. For example, the control unit 1135 may determine a distance between each pair of probes of the plurality of probes. FIG. 15 depicts an exemplary user interface stage for reviewing placed probe measurements in accordance with an embodiment. As shown, after determining a placed position 1310 of the origin probe, a placed position 1325 of the secondary probe, and/or a placed position 1330 of one or more tertiary probes, the control unit 1135 may determine a distance of each probe 1115 from every other probe 1115. While conventional systems require a user to manually measure and enter such information, the tracked locations of the probes enable these measurements to be automated by the system 1100, thereby simplifying and shortening the setup for the procedure. As shown, the user may provide input via the user interface to accept all probe positions once satisfied.

In some embodiments, a map of electric field strength across the selected target zone may be displayed to the user on the user interface via the display device 1125. For example, the ablation plan may include an electric field strength map based on the planned probe arrangement. After placing the probes and determining measurements therebetween as described, the electric field strength map may be updated to reflect the actual placement of the probes 1115. The updated electric field strength map may be displayed to the user on the display device 1125 (in a similar manner to the depiction of FIG. 10). In some embodiments, pulse information may be displayed to the user as shown, e.g., treatment parameters and/or information related to the planned series of pulses. In some embodiments, the user may modify pulse information using the input device 1130 to finalize the treatment parameters with respect to the placed probes 1115. Accordingly, additional updates may be made to the ablation plan based on the final placement of the probes 1115.

In some embodiments, the method 1200 further comprises generating a set of electrical pulses by the generator 1110 via the plurality of probes 1115 based on the ablation plan. For example, the set of electrical pulses may be generated based on the updated ablation plan to carry out the IRE procedure and ablate the target zone. In some embodiments, updates and live information may be displayed on the user interface during the ablation process as described herein.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

In some embodiments, one or more components of the system 1100 as described herein may be omitted. For example, the system 1100 may not include an imaging device 1105. However, the system 1100 may nonetheless be configured to be operatively coupled to an imaging device, i.e., the control unit 1135 may be configured to be in electrical communication with an external imaging device. Accordingly, while an imaging device may not be included in the system 1100, the system 1100 may interface with an imaging device via the control unit 1135 in order to receive one or more anatomical images of the patient.

In some embodiments, the system 1100 may prompt the user to re-place or adjust a probe 1115. For example, a probe 1115 may be poorly placed such that the ablation plan cannot be feasibly carried out even with substantial adjustments. Accordingly, an indication on the user interface may notify the user that a probe must be re-placed. In some embodiments, the control unit 1135 may assess the distance between a projected position and a placed position for a probe 1115 in order to determine whether the probe 1115 must be re-placed. For example, probes 1115 having a placed position within a predetermined threshold distance of the projected position may be acceptable and may not require repositioning. However, probes 1115 having a placed position at a distance from the projected position that is greater than the predetermined threshold distance may require repositioning. Accordingly, the system 1100 may prompt the user to reposition the probes 1115 when necessary. In some embodiments, the system 1100 may provide feedback to the user related to the re-positioning the probe, e.g., a direction and/or a distance of movement.

In some embodiments, the system 1100 may prompt the user to adjust an orientation and/or a depth of a probe 1115. In order to carry out the IRE procedure, it may be necessary to ensure that the probes 1115 are oriented substantially parallel to one another and located at substantially the same depth as one another. Accordingly, an indication on the user interface may notify the user that a probe must be adjusted to fix an orientation and/or a depth thereof. In some embodiments, the control unit 1135 may assess the orientation and/or depth of the probes based on signals from the sensors 1120. Accordingly, the system 1100 may prompt the user to adjust the orientation and/or depth of the probes 1115 when necessary.

As described herein with respect to the position of the secondary probe, the depth and/or orientation of the secondary probe may be guided based on the real-time detected depth and/or orientation. For example, the secondary probe may be displayed 1230 on display device 1125 in a first color (e.g., red) when a depth and/or orientation of the probe is inadequate. When the depth and/or orientation of the secondary probe is moved to an adequate depth and/or orientation, the first color may be changed to a second color (e.g., green) such that the secondary probe may be displayed 1230 on display device 1125 in the second color, thereby indicating adequate depth and/or orientation of the secondary probe to the user. In some embodiments, more than two colors may be used to provide feedback to the user related to the depth and/or orientation of the secondary probe. For example, a first color (e.g., red) may indicate that the orientation is poor, a second color (e.g., blue) may indicate that the depth is poor, and a third color (e.g., green) may indicate that the depth and orientation are adequate. In some embodiments, different colors may be used to indicate feedback related to the depth and/or orientation than the colors used to indicate feedback related to the position of the secondary probe as described herein.

It should be noted that the method 1200 generally relies on the placement of the origin probe to determine projected locations for subsequent probes. Accordingly, the surgeon should take particular care in placing the origin probe correctly because the system 1100 may provide less guidance for placement of such probe. However, in some embodiments, the system may display a projected position for the origin probe with respect to the anatomy of the patient and a real-time position of the origin probe in order to enable guiding the probe to a planned position from the ablation plan. In some embodiments, one or more additional sensors 1120 may be provided in the operation environment for tracking the location of the patient. For example, a sensor 1120 may be affixed to the patient to provide a reference point for the patient anatomy. Accordingly, a projected position for the origin probe may be displayed with respect to the patient anatomy based on the ablation plan in order to aid in placement of the origin probe.

In some embodiments, additional types of guidance may be provided for placement of the probes 1115. For example, after placement of an origin probe, the projected positions for all remaining probes 1115 may be displayed to a user in a variety of manners in addition to on the display 1125. For example, the projected positions for the remaining probes 1115 may be displayed on the patient with respect to the placed position of the origin probe using lasers, lights, or other types of visual indicators in order to guide placement of the remaining probes 1115. In another example, the projected positions for the remaining probes 1115 may be displayed using an augmented reality (AR) headset, whereby the projected positions are represented by AR elements virtually positioned on the patient with respect to the placed position of the origin probe in order to guide placement of the remaining probes 1115.

While the systems and methods described herein generally utilize a transverse viewpoint of the patient anatomy for planning and guidance, it should be understood that a transverse view, a sagittal view, and/or combinations thereof may be used for viewing the patient anatomy, planning locations for probes, and guiding probes to projected locations.

While the systems and methods described herein generally related to IRE procedures, it is to be understood that the present disclosure is equally applicable to other nonthermal ablation techniques, such as, reversible electroporation, electro-chemotherapy (ECT), high frequency irreversible electroporation (HFIRE), pulsed field ablation (PFA), pulsed electric fields (PEF), and the like as would be apparent to a person having an ordinary level of skill in the art.

### Data Processing Systems for Implementing Embodiments Herein

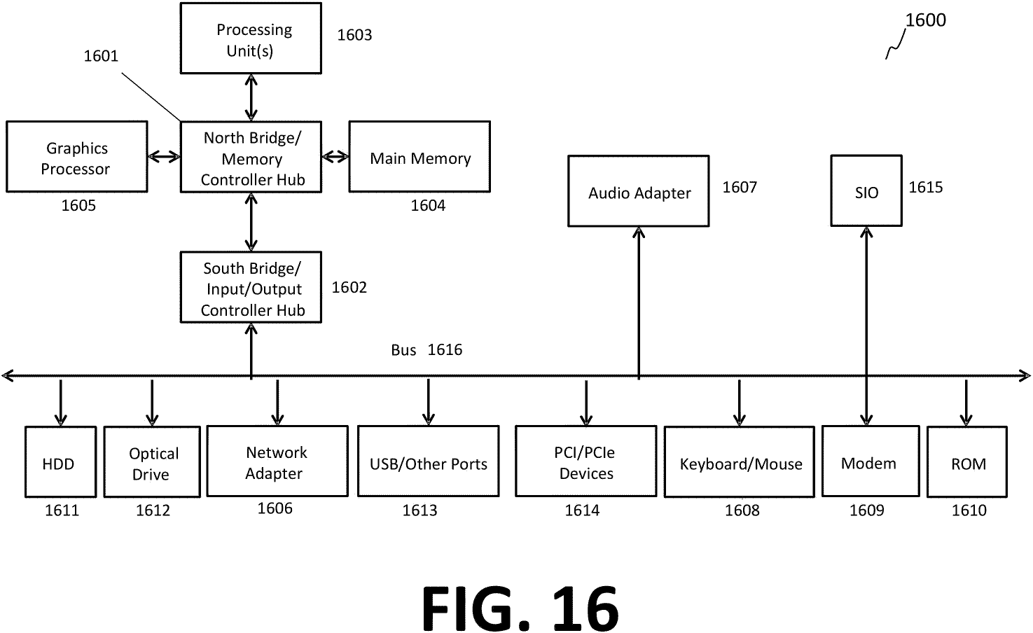

FIG. 16 illustrates a block diagram of an exemplary data processing system 1600 in which embodiments are implemented. The data processing system 1600 is an example of a computer, such as a server or client, in which computer usable code or instructions implementing the process for illustrative embodiments of the present invention are located. In some embodiments, the data processing system 1600 may be a server computing device. For example, data processing system 1600 can be implemented in a server or another similar computing device operably connected to a system 200 and/or a system 1100 as described above. The data processing system 1600 can be configured to, for example, transmit and receive information related to a patient and/or a related ablation plan with the system 200 and/or the system 1100.

In the depicted example, data processing system 1600 can employ a hub architecture including a north bridge and memory controller hub (NB/MCH) 1601 and south bridge and input/output (I/O) controller hub (SB/ICH) 1602. Processing unit 1603, main memory 1604, and graphics processor 1605 can be connected to the NB/MCH 1601. Graphics processor 1605 can be connected to the NB/MCH 1601 through, for example, an accelerated graphics port (AGP).

In the depicted example, a network adapter 1606 connects to the SB/ICH 1602. An audio adapter 1607, keyboard and mouse adapter 1608, modem 1609, read only memory (ROM) 1610, hard disk drive (HDD) 1611, optical drive (e.g., CD or DVD) 1612, universal serial bus (USB) ports and other communication ports 1613, and PCI/PCIe devices 1614 may connect to the SB/ICH 1602 through bus system 1616. PCI/PCIe devices 1614 may include Ethernet adapters, add-in cards, and PC cards for notebook computers. ROM 1610 may be, for example, a flash basic input/output system (BIOS). The HDD 1611 and optical drive 1612 can use an integrated drive electronics (IDE) or serial advanced technology attachment (SATA) interface. A super I/O (SIO) device 1615 can be connected to the SB/ICH 1602.

An operating system can run on the processing unit 1603. The operating system can coordinate and provide control of various components within the data processing system 1600. As a client, the operating system can be a commercially available operating system. An object-oriented programming system, such as the Java™ programming system, may run in conjunction with the operating system and provide calls to the operating system from the object-oriented programs or applications executing on the data processing system 1600. As a server, the data processing system 1600 can be an IBM® eServer™ System^{®} running the Advanced Interactive Executive operating system or the Linux operating system. The data processing system 1600 can be a symmetric multiprocessor (SMP) system that can include a plurality of processors in the processing unit 1603. Alternatively, a single processor system may be employed.

Instructions for the operating system, the object-oriented programming system, and applications or programs are located on storage devices, such as the HDD 1611, and are loaded into the main memory 1604 for execution by the processing unit 1603. The processes for embodiments described herein can be performed by the processing unit 1603 using computer usable program code, which can be located in a memory such as, for example, main memory 1604, ROM 1610, or in one or more peripheral devices.

A bus system 1616 can be comprised of one or more busses. The bus system 1616 can be implemented using any type of communication fabric or architecture that can provide for a transfer of data between different components or devices attached to the fabric or architecture. A communication unit such as the modem 1609 or the network adapter 1606 can include one or more devices that can be used to transmit and receive data.

Those of ordinary skill in the art will appreciate that the hardware depicted in FIG. 16 may vary depending on the implementation. Other internal hardware or peripheral devices, such as flash memory, equivalent non-volatile memory, or optical disk drives may be used in addition to or in place of the hardware depicted. Moreover, the data processing system 1600 can take the form of any of a number of different data processing systems, including but not limited to, client computing devices, server computing devices, tablet computers, laptop computers, telephone or other communication devices, personal digital assistants, and the like. Essentially, data processing system 1600 can be any known or later developed data processing system without architectural limitation.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A system for preparing an ablation procedure, the system comprising:
a generator configured to generate at least one electrical pulse for delivery to a target tissue;
a plurality of probes operably connected to the generator, the plurality of probes configured to deliver the at least one electrical pulse to the target tissue;
one or more sensors configured to indicate a position of each probe of the plurality of probes;
a display device;
a processor operably connected to the generator and the display; and
a non-transitory, computer-readable medium storing instructions that, when executed, cause the processor to:
receive an image of the target tissue,
obtain an ablation plan relating to the target tissue, the ablation plan comprising a planned probe arrangement including a planned position for each of the plurality of probes,
identify, based on the image and the planned probe arrangement, a projected position for an origin probe of the plurality of probes,
determine, based on one or more signals from the one or more sensors, a real-time position of the origin probe,
display, on the display device, the projected position and the real-time position for the origin probe superimposed over the image, and
determine a placed position of the origin probe based on the real-time position.

2. The system of claim 1, wherein the processor is operably connected to an imaging device, and
wherein the instructions to receive an image of the target tissue comprise instructions that, when executed, cause the processor to receive the image of the target tissue from the imaging device.

3. The system of claim 1, wherein the instructions, when executed, further cause the processor to:
compare the placed position of the origin probe to the projected position of the origin probe, and
modify the ablation plan based on the comparison.

4. The system of claim 3, wherein the instructions to modify the ablation plan comprise instructions that, when executed, cause the processor to update the planned position for one or more additional probes of the plurality of probes based on the comparison.

5. The system of claim 3, wherein the instructions to modify the ablation plan comprise instructions that, when executed, cause the processor to update one or more treatment parameters of the ablation plan based on the comparison.

6. The system of claim 1, further comprising one or more input devices, and wherein the instructions to obtain an ablation plan comprise instructions that, when executed, cause the processor to:
identify, based on the image, a proposed target zone comprising at least a portion of the target tissue,
display, on the display device, the proposed target zone superimposed over the anatomical image,
receive, by the one or more input devices, target feedback related to the proposed target zone,
modify the proposed target zone based on the target feedback to define a selected target zone, and
generate the ablation plan, wherein the ablation plan comprises the selected target zone.

7. The system of claim 6, wherein the instructions, when executed, further cause the processor to:
measure one or more distances in the selected target zone based on the image, and
define a location of the selected target zone based on the image,
wherein the ablation plan is based on the one or more distances and the location of the selected target zone.

8. The system of claim 6, wherein the instructions to generate the ablation plan comprise instructions that, when executed, further cause the processor to:
identify, based on the selected target zone, a proposed probe arrangement comprising a proposed position for each of the plurality of probes,
display, on the display device, the proposed probe arrangement superimposed over the image,
receive, by the one or more input devices, probe feedback related to the proposed probe arrangement,
modify the proposed probe arrangement based on the probe feedback to define a selected probe arrangement, and
update the ablation plan based on the selected probe arrangement, wherein the planned probe arrangement of the ablation plan comprises the selected probe arrangement.

9. The system of claim 8, wherein the probe feedback comprises one or more of:
an indication of one or more of the plurality of probes to be removed from the proposed probe arrangement;
an indication to add one or more additional probes to the plurality of probes in the proposed probe arrangement;
an indication to adjust the proposed position of one or more of the plurality of probes; and
an indication of approval of the proposed probe arrangement.

10. The system of claim 8, wherein the ablation plan further comprises a planned series of pulses to be emitted between the plurality of probes,
wherein the instructions to update the ablation plan based on the selected probe arrangement comprise instructions that, when executed, cause the processor to update the planned series of pulses based on the selected probe arrangement.

11. An ablation system comprising a non-transitory, computer-readable medium storing instructions that, when executed by a processor, cause the processor to:
receive an image of a target tissue;
obtain a planned probe arrangement including a planned position for each of a plurality of probes;
identify, based on the image and the planned probe arrangement, a projected position for a first probe of the plurality of probes;
determine, based on one or more signals from one or more sensors, a real-time position of the first probe;
display, via a display device, the projected position and the real-time position for the first probe superimposed over the image; and
determine a placed position of the first probe based on the real-time position.

12. The ablation system of claim 11, wherein the instructions, when executed, further cause the processor to:
compare the placed position of the first probe to the projected position of the first probe, and
modify a treatment plan based on the comparison.

13. The system of claim 12, wherein the instructions to modify the treatment plan comprise instructions that, when executed, cause the processor to update the planned position for one or more additional probes of the plurality of probes based on the comparison.

14. The system of claim 12, wherein the instructions to modify the treatment plan comprise instructions that, when executed, cause the processor to update one or more treatment parameters of the treatment plan based on the comparison.

15. A system for planning an ablation procedure, the system comprising:
a display device;
a processor operably connected to the display device and the one or more input devices; and
a non-transitory, computer-readable medium storing instructions that, when executed, cause the processor to:
receive an image of a patient comprising a target tissue,
receive, by one or more input devices, target feedback related to the identification of a target zone,
identify a planned position for a first probe of a plurality of probes relative to the target zone,
determine, based on one or more signals from a sensor, a real-time position of the first probe,
display, on the display device, the planned position and the real-time position for the first probe superimposed over the image of the target tissue and the target zone, and
determine a placed position of the first probe based on the real-time position.

16. The system of claim 15, further comprising:
a generator configured to generate at least one electrical pulse for delivery to a target tissue; and
the plurality of probes operably connected to the generator, wherein the plurality of probes are configured to deliver the at least one electrical pulse to the target tissue to irreversibly electroporate substantially all of the target tissue in the target zone.

17. The system of claim 15, wherein the sensor comprises an electromagnetic sensor.

18. The system of claim 17, further comprising the electromagnetic sensor and an electromagnetic generator operably connected to the processor.

19. The system of claim 18, wherein the electromagnetic generator is arranged and configured to generate an electromagnetic field in a region including the target tissue.

20. The system of claim 19, wherein the instructions to determine a real-time position of the first probe comprise instructions that, when executed, cause the processor to:
receive the one or more signals from the electromagnetic sensor, wherein the one or more signals are indicative of a characteristic of the electromagnetic field sensed by the sensor, and
determine a location of the electromagnetic sensor based on the one or more signals, wherein the location of the electromagnetic sensor is indicative of the real-time position of the first probe.
